(19) United States
(12) Patent Application Publication
(10) Pub. No.: US 2024/0423992 A1
(43) Pub. Date: Dec. 26, 2024

(54) BIOMARKERS FOR TREATING LIVER DISORDERS WITH THR-B AGONISTS AND RELATED USES

(71) Applicant: Madrigal Pharmaceuticals, Inc., West Conshohocken, PA (US)

(72) Inventor: Rebecca TAUB, Villanova, PA (US)

(21) Appl. No.: 18/748,811

(22) Filed: Jun. 20, 2024

Related U.S. Application Data
(60) Provisional application No. 63/509,740, filed on Jun. 22, 2023.
(60) Provisional application No. 63/578,163, filed on Aug. 23, 2023.

(51) Int. Cl. A61K 31/53; A61P 1/16; G01N 33/78

(52) U.S. Cl. CPC A61K 31/53; A61P 1/16; G01N 33/78; G01N 2333/59; G01N 2800/085; G01N 2800/52

(57) ABSTRACT

The present disclosure provides a method of treating or preventing a liver disorder (e.g., NASH) in a subject with a THR-β agonist, using a biomarker test (e.g., to determine a dose of the THR-β agonist).

### CROSS REFERENCE TO RELATED APPLICATIONS

This application claims the benefit of U.S. Provisional Application Nos. 63/509,740, filed Jun. 22, 2023, and 63/578,163, filed Aug. 23, 2023, the disclosures of which are incorporated herein by reference in their entireties.

### BACKGROUND

Non-alcoholic steatohepatitis (NASH) is the most common chronic liver disease in the United States. NASH is a fatty inflammation of the liver and a major cause of cirrhosis, fibrosis and liver failure. The disease is progressive, starting as steatosis or nonalcoholic fatty liver disease (NAFLD), progressing to an inflamed fatty liver (NASH), and eventually leading to cirrhosis and fibrosis. The disease is generally asymptomatic until severe liver impairment occurs.

The prevalence of NAFLD in the U.S. population is about 20-23%, and may be as high as 33%, and the prevalence of NASH in the U.S. population is about 2-3%. Some NASH patients will progress to late-stage disease: approximately 15-50% of NASH patients progress to severe fibrosis, and approximately 7-16% progress to cirrhosis. The rate of liver-specific mortality in NASH cirrhotics is approximately 10% per decade.

Currently, no specific therapies for NASH exist. There is a need to develop new methods of treating NASH. The present disclosure addresses this need.

### SUMMARY

In some aspects, the present disclosure provides a method of treating or preventing a liver disorder in a subject, comprising:
(a) performing a baseline biomarker test on a biological sample obtained from the subject;
(b) administering a first dose of a thyroid hormone receptor-β (THR-β) agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some aspects, the present disclosure provides a THR-β agonist for use in treating or preventing a liver disorder in a subject, wherein the treating or preventing comprises:
(a) performing a baseline biomarker test on a biological sample obtained from the subject;
(b) administering a first dose of the THR-β agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some aspects, the present disclosure provides use of a THR-β agonist in the manufacture of a medicament for treating or preventing a liver disorder in a subject, wherein the treating or preventing comprises:
(a) performing a baseline biomarker test on a biological sample obtained from the subject;
(b) administering a first dose of the THR-β agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some aspects, the present disclosure provides a method of treating or preventing a liver disorder in a subject, comprising:
(a) administering a low-age dose of a THR-β agonist to the subject, wherein the subject has an age younger than a dose-adjustment age threshold; and/or
(b) administering a high-age dose of the THR-β agonist to the subject, wherein the subject has an age at a dose-adjustment age threshold or older.

In some aspects, the present disclosure provides a THR-β agonist for use in treating or preventing a liver disorder in a subject, wherein the treating or preventing comprises:
(a) administering a low-age dose of a THR-β agonist to the subject, wherein the subject has an age younger than a dose-adjustment age threshold; and/or
(b) administering a high-age dose of the THR-B agonist to the subject, wherein the subject has an age at a dose-adjustment age threshold or older.

In some aspects, the present disclosure provides use of a THR-β agonist in the manufacture of a medicament for treating or preventing a liver disorder in a subject, wherein the treating or preventing comprises:
(a) administering a low-age dose of a THR-β agonist to the subject, wherein the subject has an age younger than a dose-adjustment age threshold; and/or
(b) administering a high-age dose of the THR-B agonist to the subject, wherein the subject has an age at a dose-adjustment age threshold or older.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the disclosure will be apparent from the following detailed description and claims.

### BRIEF DESCRIPTION OF THE DRAWINGS

FIG. 1 is a graph showing the SHBG level in subject upon administering resmetirom or placebo over 48 weeks.

FIG. 2 is a graph showing least squares (LS) mean change from baseline (95% confidence interval (CI)) in hepatic fat (measured by magnetic resonance imaging-proton density fat fraction (MRI-PDFF)) at Week 52 in patient subgroups of the double-blind 100 mg resmetirom and double-blind 80 mg resmetirom arms. Marker shows median. High SHBG, population with ≥120% increase from baseline in SHBG.

FIGS. 3A and 3B are a set of graphs showing least squares (LS) mean change from baseline (95% confidence interval (CI)) in PDFF (a) and liver volume (b) at Week 52 in patient subgroups of the open-label 100 mg resmetirom arm. Marker shows median. High SHBG, population (˜two-thirds) with ≥120% increase from baseline in SHBG.

FIGS. 4A and 4B are a set of graphs showing NASH resolution and fribrosis improvement of various patient subgroup treated with resmetirom as compared to placebo (T2D=Type 2 Diabetes; NAS=NAFLD Activity Score).

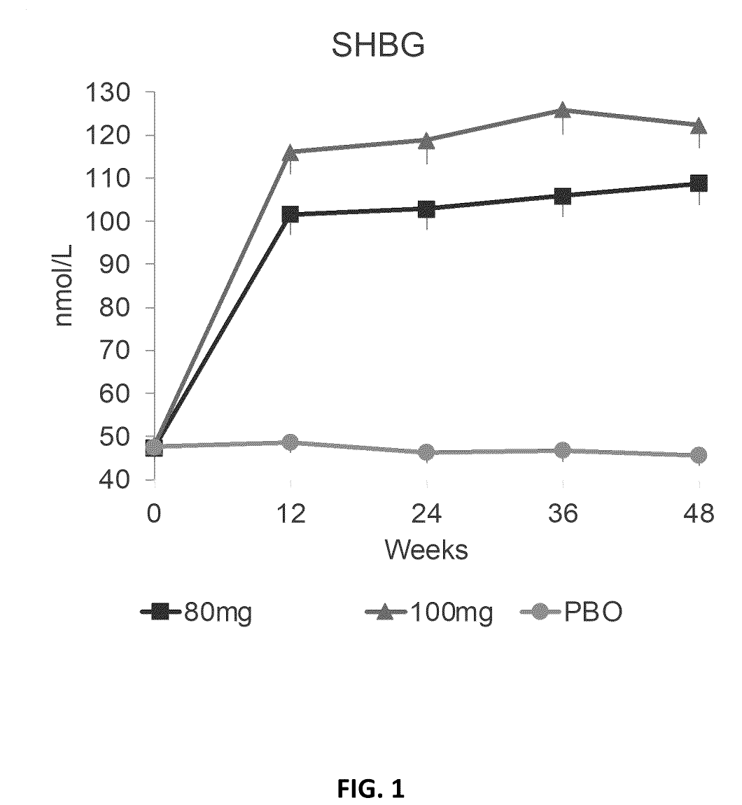

### DETAILED DESCRIPTION

### Compound A

As used herein, the term “Compound A” refers to resmetirom, a prodrug thereof, or a pharmaceutically acceptable salt thereof. It is understood that Compound A could be present in a morphic form (e.g., an amorphous form, or a crystalline form) of a mixture of morphic forms.

In some embodiments, Compound A is resmetirom.

In some embodiments, Compound A is a morphic form of resmetirom.

In some embodiments, Compound A is a crystalline form of resmetirom.

In some embodiments, Compound A is a morphic form (e.g., crystalline form) of resmetirom as described in PCT Pub. No. WO/2014/043706 (incorporated herein by reference).

In some embodiments, Compound A is Form I of resmetirom.

In some embodiments, Compound A is a morphic form of resmetirom, wherein the form is characterized by an X-ray powder diffraction pattern including signals (e.g., peaks) at about 10.5, about 18.7, about 22.9, about 23.6, and about 24.7 degrees 2θ. In some embodiments, the X-ray powder diffraction pattern further includes peaks at about 8.2, about 11.2, about 15.7, about 16.4, about 17.7, about 30.0, and about 32.2 degrees 2θ.

In some embodiments, Compound A is a morphic form (e.g., crystalline form) of resmetirom as described in PCT Pub. No. WO/2022/086894 (incorporated herein by reference).

In some embodiments, Compound A is a prodrug of resmetirom.

In some embodiments, Compound A is a morphic form of a prodrug of resmetirom.

In some embodiments, Compound A is a crystalline form of a prodrug of resmetirom.

In some embodiments, Compound A is a pharmaceutically acceptable salt of resmetirom.

In some embodiments, Compound A is a morphic form of a pharmaceutically acceptable salt of resmetirom.

In some embodiments, Compound A is a crystalline form of a pharmaceutically acceptable salt of resmetirom.

In some embodiments, Compound A is a morphic form (e.g., crystalline form) of a pharmaceutically acceptable salt of resmetirom as described in PCT Pub. No. WO/2020/010068 (incorporated herein by reference).

In some embodiments, Compound A has a purity of at least about 80%, at least about 85%, at least about 90%, at least about 95%, at least about 96%, at least about 97%, at least about 98%, at least about 99%, at least about 99.5%, or at least about 99.9%.

As used herein, the term “resmetirom” refers to a compound having the following structure:

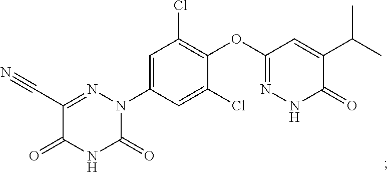 [SMILES: CC(C)c1cc(Oc2c(Cl)cc(-n3nc(C#N)c(=O)[nH]c3=O)cc2Cl)n[nH]c1=O]

and resmetirom may be identified by CAS No. 920509-32-6, by code name MGL-3196, or by IUPAC name 2-[3,5-dichloro-4-[(6-oxo-5-propan-2-yl-1H-pyridazin-3-yl)oxy]phenyl]-3,5-dioxo-1,2,4-triazine-6-carbonitrile.

Various forms of Compound A, and isomers, salts, and prodrugs thereof, are described in PCT Pub. Nos. WO/2020/010068 and WO/2020/010068 (each of which is incorporated herein by reference). Additional forms of Compound A, and isomers, salts, and prodrugs thereof, are described in PCT Pub. Nos. WO/2022/086894, WO/2022/052822, WO/2021/129465, and WO/2021/063367 (each of which is incorporated herein by reference).

All references to Compound A herein include all pharmaceutically acceptable salts and all solvates and alternative physical forms thereof, unless otherwise stated. All doses recited herein for Compound A are based on the molecular weight of Compound A itself, rather than the pharmaceutically acceptable salt, hydrate of solvate thereof or any excipients in the composition, unless otherwise stated.

### Methods the Present Disclosure

In some aspects, the present disclosure provides a method of treating or preventing a liver disorder in a subject, comprising:
(a) performing a baseline biomarker test on a biological sample obtained from the subject;
(b) administering a first dose of a thyroid hormone receptor-β (THR-β) agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some aspects, the present disclosure provides a method of treating a liver disorder in a subject, comprising:
(a) performing a baseline biomarker test on a biological sample obtained from the subject;
(b) administering a first dose of a thyroid hormone receptor-β (THR-β) agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some aspects, the present disclosure provides a THR-β agonist for use in treating or preventing a liver disorder in a subject, wherein the treating or preventing comprises:
(a) performing a baseline biomarker test on a biological sample obtained from the subject;
(b) administering a first dose of the THR-β agonist to the subject;
(c) performing a benchmark biomarker test on a biological sample obtained from the subject;
(d) performing a result assessment, thereby determining a second dose of the THR-B agonist; and
(e) administering the second dose of the THR-B agonist to the subject.

In some aspects, the present disclosure provides a THR-β agonist for use in treating a liver disorder in a subject, wherein the treating comprises:
(a) performing a baseline biomarker test on a biological sample obtained from the subject;
(b) administering a first dose of the THR-β agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some aspects, the present disclosure provides use of a THR-β agonist in the manufacture of a medicament for treating or preventing a liver disorder in a subject, wherein the treating or preventing comprises:
(a) performing a baseline biomarker test on a biological sample obtained from the subject;
(b) administering a first dose of the THR-β agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some aspects, the present disclosure provides use of a THR-β agonist in the manufacture of a medicament for treating a liver disorder in a subject, wherein the treating comprises:
(a) performing a baseline biomarker test on a biological sample obtained from the subject;
(b) administering a first dose of the THR-β agonist to the subject;
(c) performing a benchmark biomarker test on a biological sample obtained from the subject;
(d) performing a result assessment, thereby determining a second dose of the THR-B agonist; and
(e) administering the second dose of the THR-β agonist to the subject.

In some embodiments, the THR-β agonist is a selective THR-β agonist.

In some embodiments, the THR-β agonist is sobetirome (GC-1), eprotirome (KB2115), VK2809 (MB08711), Sob-AM2, IS25, or TG68. Additional suitable THR-β agonists are described in, e.g., Saponaro et al. (Selective Thyroid Hormone Receptor-Beta (TRβ) Agonists: New Perspectives for the Treatment of Metabolic and Neurodegenerative Disorders. Front Med. 7:331 (2020); incorporated by reference).

In some embodiments, the THR-β agonist is Compound A.

In some aspects, the present disclosure provides a method of treating or preventing a liver disorder in a subject, comprising:
(a) performing a baseline biomarker test on a biological sample obtained from the subject;
(b) administering a first dose of Compound A to the subject;
(c) performing a benchmark biomarker test on a biological sample obtained from the subject;
(d) performing a result assessment, thereby determining a second dose of Compound A; and
(e) administering the second dose of Compound A to the subject.

In some aspects, the present disclosure provides a method of treating a liver disorder in a subject, comprising:
(a) performing a baseline biomarker test on a biological sample obtained from the subject;
(b) administering a first dose of Compound A to the subject;
(c) performing a benchmark biomarker test on a biological sample obtained from the subject;
(d) performing a result assessment, thereby determining a second dose of Compound A; and
(e) administering the second dose of Compound A to the subject.

In some aspects, the present disclosure provides Compound A for use in treating or preventing a liver disorder in a subject, wherein the treating or preventing comprises:
(a) performing a baseline biomarker test on a biological sample obtained from the subject;
(b) administering a first dose of Compound A to the subject;
(c) performing a benchmark biomarker test on a biological sample obtained from the subject;
(d) performing a result assessment, thereby determining a second dose of Compound A; and
(e) administering the second dose of Compound A to the subject.

In some aspects, the present disclosure provides Compound A for use in treating a liver disorder in a subject, wherein the treating comprises:
(a) performing a baseline biomarker test on a biological sample obtained from the subject;
(b) administering a first dose of Compound A to the subject;
(c) performing a benchmark biomarker test on a biological sample obtained from the subject;
(d) performing a result assessment, thereby determining a second dose of Compound A; and
(e) administering the second dose of Compound A to the subject.

In some aspects, the present disclosure provides use of Compound A in the manufacture of a medicament for treating or preventing a liver disorder in a subject, wherein the treating or preventing comprises:
(a) performing a baseline biomarker test on a biological sample obtained from the subject;
(b) administering a first dose of Compound A to the subject;
(c) performing a benchmark biomarker test on a biological sample obtained from the subject;
(d) performing a result assessment, thereby determining a second dose of Compound A; and
(e) administering the second dose of Compound A to the subject.

In some aspects, the present disclosure provides use of Compound A in the manufacture of a medicament for treating a liver disorder in a subject, wherein the treating comprises:
(a) performing a baseline biomarker test on a biological sample obtained from the subject;
(b) administering a first dose of Compound A to the subject;
(c) performing a benchmark biomarker test on a biological sample obtained from the subject;
(d) performing a result assessment, thereby determining a second dose of Compound A; and
(e) administering the second dose of Compound A to the subject.

### Additional Methods

In some aspects, the present disclosure provides a method, comprising:
(c) performing a benchmark biomarker test on a biological sample obtained from a subject; and
(d) performing a result assessment, thereby determining a second dose of a THR-β agonist.

In some aspects, the present disclosure provides a method, comprising
(c) performing a benchmark biomarker test on a biological sample obtained from a subject, wherein a baseline biomarker test is previously performed on a biological sample obtained from the subject; and
(d) performing a result assessment, thereby determining a second dose of a THR-β agonist.

In some aspects, the present disclosure provides a method, comprising:
(c) performing a benchmark biomarker test on a biological sample obtained from a subject, wherein a baseline biomarker test is previously performed on a biological sample obtained from the subject, and wherein a first dose of the THR-B agonist is previously administered to the subject; and
(d) performing a result assessment, thereby determining a second dose of a THR-B agonist.

In some embodiments, the method further comprises:
(e) administering the second dose of the THR-β agonist to the subject.

In some aspects, the present disclosure provides a method, comprising:
(c) performing a benchmark biomarker test on a biological sample obtained from a subject; and
(d) performing a result assessment, thereby determining a second dose of Compound A.

In some aspects, the present disclosure provides a method, comprising
(c) performing a benchmark biomarker test on a biological sample obtained from a subject, wherein a baseline biomarker test is previously performed on a biological sample obtained from the subject; and
(d) performing a result assessment, thereby determining a second dose of Compound A.

In some aspects, the present disclosure provides a method, comprising:
(c) performing a benchmark biomarker test on a biological sample obtained from a subject, wherein a baseline biomarker test is previously performed on a biological sample obtained from the subject, and wherein a first dose of Compound A is previously administered to the subject; and
(d) performing a result assessment, thereby determining a second dose of Compound A.

In some embodiments, the method further comprises:
(e) administering the second dose of Compound A to the subject.

In some aspects, the present disclosure provides a composition for use in a method described herein.

In some aspects, the present disclosure provides a kit, comprising: a composition for use in a method described herein; and an instruction of performing the method.

In some aspects, the present disclosure provides a device for use in a method described herein.

In some aspects, the present disclosure provides a kit, comprising: a device for use in a method described herein; and an instruction of performing the method.

### Diseases and Subjects

In some embodiments, the liver disorder is a fatty liver disease.

In some embodiments, the liver disorder is non-alcoholic fatty liver disease (NAFLD).

In some embodiments, the liver disorder is non-alcoholic steatohepatitis (NASH).

It is understood that, in NASH, the fat accumulation may be associated with varying degrees of inflammation (hepatitis) and/or scarring (fibrosis) of the liver.

In some embodiments, the liver disorder is NASH with liver fibrosis.

In some embodiments, the subject is a human.

In some embodiments, the subject is a human with an age of 18 years or older.

In some embodiments, the subject is a male.

In some embodiments, the subject is a female.

### Baseline Biomarker Tests

In some embodiments, the baseline biomarker test is performed before administering the first dose of Compound A.

In some embodiments, the baseline biomarker test is performed at about one minute, about two minutes, about three minutes, about four minutes, about five minutes, about 10 minutes, about 20 minutes, about 30 minutes, about 40 minutes, about 50 minutes, or about 60 minutes, before administering the first dose of Compound A.

In some embodiments, the baseline biomarker test is performed at about one hour, about two hours, about three hours, about four hours, about five six hours, about seven hours, about eight hours, about nine hours, about 10 hours, about 11 hours, about 12 hours, about 18 hours, or about 24 hours, before administering the first dose of Compound A.

In some embodiments, the baseline biomarker test is performed at about one day, about two days, about three days, about four days, about five six days, or about seven days, before administering the first dose of Compound A.

In some embodiments, the baseline biomarker test comprises measuring the expression level of a biomarker.

In some embodiments, the baseline biomarker test comprises measuring the expression level of a sex hormone.

In some embodiments, the sex hormone is estradiol, follicle-stimulating hormone (FSH), luteinizing hormone (LH), testosterone, free testosterone, sex hormone-binding globulin (SHBG), or any combination thereof.

In some embodiments, the baseline biomarker test comprises measuring the expression level of estradiol, FSH, LH, testosterone, free testosterone, SHBG, or any combination thereof.

In some embodiments, the sex hormone is SHBG.

In some embodiments, the baseline biomarker test comprises measuring the expression level of SHBG.

In some embodiments, the baseline biomarker test further comprises measuring the expression level of a thyroid hormone.

In some embodiments, the thyroid hormone is free triiodothyronine (FT3), reverse triiodothyronine (RT3), bound T3, total T3, free thyroxine (FT4), bound T4, total T4, or thyroid-stimulating hormone (TSH), thyroxine binding globulin (TBG), or any combination thereof. It is understood that total T3 refers to the sum of bound T3 and free T3, and that total T4 refers to the sum of bound T4 and free T4.

In some embodiments, the thyroid hormone is FT3, RT3, FT4, TSH, TBG, or any combination thereof.

In some embodiments, the baseline biomarker test further comprises measuring the expression level of FT3, RT3, FT4, TSH, TBG, or any combination thereof.

In some embodiments, the thyroid hormone is FT4.

In some embodiments, the baseline biomarker test further comprises measuring the expression level of FT4.

In some embodiments, the baseline biomarker test comprises measuring: (i) the expression level of a sex hormone, and (ii) the expression level of a thyroid hormone.

In some embodiments, the baseline biomarker test comprises measuring: (i) the expression level of estradiol, FSH, LH, testosterone, free testosterone, SHBG, or any combination thereof, and (ii) the expression level of FT3, RT3, FT4, TSH, TBG, or any combination thereof.

In some embodiments, the baseline biomarker test comprises measuring: (i) the expression level of SHBG, and (ii) the expression level of FT4.

### First Dose Administrations

In some embodiments, the first dose of Compound A is administered to the subject daily.

In some embodiments, the first dose of Compound A is administered to the subject once daily.

In some embodiments, the first dose of Compound A is administered to the subject two or more times daily.

In some embodiments, the first dose of Compound A is a daily dose of:
about 40±10 mg, about 40±5 mg, about 40±4 mg, about 40±3 mg, about 40±2 mg, or about 40±1 mg (e.g., about 40 mg);
about 60±20 mg, about 60±10 mg, about 60±5 mg, about 60±4 mg, about 60±3 mg, about 60±2 mg, or about 60±1 mg (e.g., about 60 mg);
about 80±20 mg, about 80±10 mg, about 80±5 mg, about 80±4 mg, about 80±3 mg, about 80±2 mg, or about 80±1 mg (e.g., about 80 mg);
about 100±30 mg, about 100±20 mg, about 100±10 mg, about 100±5 mg, about 100±4 mg, about 100±3 mg, about 100±2 mg, or about 100±1 mg (e.g., about 100 mg); or about 120±40 mg, about 120±30 mg, about 120±20 mg, about 120±10 mg, about 120±5 mg, about 120±4 mg, about 120±3 mg, about 120±2 mg, or about 120±1 mg (e.g., about 120 mg).

In some embodiments, the first dose of Compound A is a daily dose of:
about 60±20 mg, about 60±10 mg, about 60±5 mg, about 60±4 mg, about 60±3 mg, about 60±2 mg, or about 60±1 mg (e.g., about 60 mg);
about 80±20 mg, about 80±10 mg, about 80±5 mg, about 80±4 mg, about 80±3 mg, about 80±2 mg, or about 80±1 mg (e.g., about 80 mg); or about 100±30 mg, about 100±20 mg, about 100±10 mg, about 100±5 mg, about 100±4 mg, about 100±3 mg, about 100±2 mg, or about 100±1 mg (e.g., about 100 mg).

In some embodiments, the first dose of Compound A is a daily dose of about 60±20 mg, about 60±10 mg, about 60±5 mg, about 60±4 mg, about 60±3 mg, about 60±2 mg, or about 60±1 mg (e.g., about 60 mg).

In some embodiments, the first dose of Compound A is a daily dose of about 80±20 mg, about 80±10 mg, about 80±5 mg, about 80±4 mg, about 80±3 mg, about 80±2 mg, or about 80±1 mg (e.g., about 80 mg).

In some embodiments, the first dose of Compound A is a daily dose of about 100±30 mg, about 100±20 mg, about 100±10 mg, about 100±5 mg, about 100±4 mg, about 100±3 mg, about 100±2 mg, or about 100±1 mg (e.g., about 100 mg).

In some embodiments, a pharmaceutical composition comprising the first dose of Compound A is administered to the subject. In some embodiments, the pharmaceutical composition is an oral formulation (e.g., a tablet).

It is understood that the first dose of Compound A may be administered to the subject for any period of time, e.g., until the second dose of Compound A is determined.

In some embodiments, the first dose of Compound A is administered to the subject for about a week or longer, about two weeks or longer, about three weeks or longer, about one month or longer, about two months or longer, about three months or longer, about four months or longer, about five months or longer, about six months or longer, about seven months or longer, about eight months or longer, about nine months or longer, about ten months or longer, about 11 months or longer, or about 12 months or longer.

In some embodiments, the first dose of Compound A is administered to the subject for about one year or longer, for about two years or longer, for about three years or longer, for about four years or longer, for about five years or longer, for about six years or longer, for about seven years or longer, for about eight years or longer, for about nine years or longer, or for about ten years or longer.

In some embodiments, the first dose of Compound A is administered to the subject for about a week, about two weeks, about three weeks, about one month, about two months, about three months, about four months, about five months, about six months, about seven months, about eight months, about nine months, about ten months, about 11 months, or about 12 months.

In some embodiments, the first dose of Compound A is administered to the subject for about one year, for about two years, for about three years, for about four years, for about five years, for about six years, for about seven years, for about eight years, for about nine years, or for about ten years.

It is understood that one or more additional biomarker tests may be performed prior to the benchmark biomarker test. In some embodiments, the first dose of Compound A is administered to the subject until the second dose of Compound A is determined.

### Benchmark Biomarker Tests

In some embodiments, the benchmark biomarker test is performed after administering the first dose of Compound A.

In some embodiments, the benchmark biomarker test is performed at about one minute, about two minutes, about three minutes, about four minutes, about five minutes, about 10 minutes, about 20 minutes, about 30 minutes, about 40 minutes, about 50 minutes, or about 60 minutes, after administering the first dose of Compound A.

In some embodiments, the benchmark biomarker test is performed at about one hour, about two hours, about three hours, about four hours, about five six hours, about seven hours, about eight hours, about nine hours, about 10 hours, about 11 hours, about 12 hours, about 18 hours, or about 24 hours, after administering the first dose of Compound A.

In some embodiments, the benchmark biomarker test is performed at about one day, about two days, about three days, about four days, about five six days, or about seven days, after administering the first dose of Compound A.

In some embodiments, the benchmark biomarker test comprises measuring the expression level of a biomarker.

In some embodiments, the benchmark biomarker test comprises measuring the expression level of a sex hormone.

In some embodiments, the benchmark biomarker test comprises measuring the expression level of estradiol, FSH, LH, testosterone, free testosterone, SHBG, or any combination thereof.

In some embodiments, the benchmark biomarker test comprises measuring the expression level of SHBG.

In some embodiments, the benchmark biomarker test further comprises measuring the expression level of a thyroid hormone.

In some embodiments, the benchmark biomarker test comprises measuring the expression level of FT3, RT3, FT4, TSH, TBG, or any combination thereof.

In some embodiments, the benchmark biomarker test further comprises measuring the expression level of FT4.

In some embodiments, the benchmark biomarker test comprises measuring: (i) the expression level of a sex hormone, and (ii) the expression level of a thyroid hormone.

In some embodiments, the benchmark biomarker test comprises measuring: (i) the expression level of estradiol, FSH, LH, testosterone, free testosterone, SHBG, or any combination thereof, and (ii) the expression level of FT3, RT3, FT4, TSH, TBG, or any combination thereof.

In some embodiments, the benchmark biomarker test comprises measuring: (i) the expression level of SHBG, and (ii) the expression level of FT4.

### Second Dose Determinations

In some embodiments, the result assessment comprises comparing the result of the benchmark biomarker test and the result of the baseline biomarker test.

Expression Level of the Sex Hormone (e.g., SHBG):

In some embodiments, the benchmark biomarker test results in a higher expression level of the sex hormone (e.g., SHBG) as compared to the baseline biomarker test.

In some embodiments, the result assessment comprises:
(i) determining that the second dose is lower than the first dose (e.g., by about 20 mg), if the benchmark biomarker test results in an expression level of the sex hormone (e.g., SHBG) at a high exposure threshold or higher, as compared to the baseline biomarker test.

In some embodiments, the result assessment comprises:
(ii) determining that the second dose is higher than the first dose, if the benchmark biomarker test results in an expression level of the sex hormone (e.g., SHBG) lower than a target engagement threshold, as compared to the baseline biomarker test.

In some embodiments, the result assessment comprises:
(iii) determining that the second dose is the same as the first dose, if the benchmark biomarker test results in an expression level of the sex hormone (e.g., SHBG) lower than the high exposure threshold, and at the target engagement threshold or higher, as compared to the baseline biomarker test.

In some embodiments, the result assessment comprises:
(i) determining that the second dose is lower than the first dose (e.g., by about 20 mg), if the benchmark biomarker test results in an expression level of the sex hormone (e.g., SHBG) at a high exposure threshold or higher, as compared to the baseline biomarker test;
(ii) determining that the second dose is higher than the first dose, if the benchmark biomarker test results in an expression level of the sex hormone (e.g., SHBG) lower than a target engagement threshold, as compared to the baseline biomarker test; and/or
(iii) determining that the second dose is the same as the first dose, if the benchmark biomarker test results in an expression level of the sex hormone (e.g., SHBG) lower than the high exposure threshold, and at the target engagement threshold or higher, as compared to the baseline biomarker test.

In some embodiments, the high exposure threshold for the expression level of the sex hormone (e.g., SHBG) is about 350% increase, about 360% increase, about 370% increase, about 380% increase, about 390% increase, about 400% increase, about 410% increase, about 420% increase, about 430% increase, about 440% increase, about 450% increase, about 460% increase, about 470% increase, about 480% increase, about 490% increase, about 500% increase, about 510% increase, about 520% increase, about 530% increase, about 540% increase, about 550% increase, about 560% increase, about 570% increase, about 580% increase, about 590% increase, or about 600% increase, as compared to the baseline biomarker test.

In some embodiments, the target engagement threshold for the expression level of the sex hormone (e.g., SHBG) is about 40% increase, about 50% increase, about 60% increase, about 70% increase, about 80% increase, about 90% increase, about 100% increase, about 110% increase, about 120% increase, about 130% increase, about 140% increase, about 150% increase, about 160% increase, about 170% increase, about 180% increase, about 190% increase, about 200% increase, about 210% increase, about 220% increase, about 230% increase, about 240% increase, about 250% increase, about 260% increase, about 270% increase, about 280% increase, about 290% increase, or about 300% increase, as compared to the baseline biomarker test.

In some embodiments, the benchmark biomarker test results in an expression level of SHBG that is higher, as compared to the baseline biomarker test, by about 350% or greater, about 360% or greater, about 370% or greater, about 380% or greater, about 390% or greater, about 400% or greater, about 410% or greater, about 420% or greater, about 430% or greater, about 440% or greater, about 450% or greater, about 460% or greater, about 470% or greater, about 480% or greater, about 490% or greater, about 500% or greater, about 510% or greater, about 520% or greater, about 530% or greater, about 540% or greater, about 550% or greater, about 560% or greater, about 570% or greater, about 580% or greater, about 590% or greater, or about 600% or greater; and the second dose is determined to be lower than the first dose by about 5 mg, about 10 mg, about 15 mg, about 20 mg, about 25 mg, about 30 mg, about 35 mg, or about 40 mg.

In some embodiments, the benchmark biomarker test results in an expression level of SHBG that is higher, as compared to the baseline biomarker test, by about 40% or less, about 50% or less, about 60% or less, about 70% or less, about 80% or less, about 90% or less, about 100% or less, about 110% or less, about 120% or less, about 130% or less, about 140% or less, about 150% or less, about 160% or less, about 170% or less, about 180% or less, about 190% or less, about 200% or less, about 210% or less, about 220% or less, about 230% or less, about 240% or less, about 250% or less, about 260% or less, about 270% or less, about 280% or less, about 290% or less, or about 300% or less; and the second dose is determined to be higher than the first dose by about 5 mg, about 10 mg, about 15 mg, about 20 mg, about 25 mg, about 30 mg, about 35 mg, or about 40 mg.

### Expression Level of the Thyroid Hormone (e.g., FT4):

In some embodiments, the benchmark biomarker test results in a lower expression level of the thyroid hormone (e.g., FT4) as compared to the baseline biomarker test.

In some embodiments, the result assessment comprises:
(i) determining that the second dose is lower than the first dose (e.g., by about 20 mg), if the benchmark biomarker test results in an expression level of the thyroid hormone (e.g., FT4) at a high exposure threshold or lower, as compared to the baseline biomarker test.

In some embodiments, the result assessment comprises:
(ii) determining that the second dose is higher than the first dose, if the benchmark biomarker test results in an expression level of the thyroid hormone (e.g., FT4) higher than a target engagement threshold, as compared to the baseline biomarker test.

In some embodiments, the result assessment comprises:
(iii) determining that the second dose is the same as the first dose, if the benchmark biomarker test results in an expression level of the thyroid hormone (e.g., FT4) higher than the high exposure threshold, and at the target engagement threshold or lower, as compared to the baseline biomarker test.

In some embodiments, the result assessment comprises:
(i) determining that the second dose is lower than the first dose (e.g., by about 20 mg), if the benchmark biomarker test results in an expression level of the thyroid hormone (e.g., FT4) at a high exposure threshold or lower, as compared to the baseline biomarker test;
(ii) determining that the second dose is higher than the first dose, if the benchmark biomarker test results in an expression level of the thyroid hormone (e.g., FT4) higher than a target engagement threshold, as compared to the baseline biomarker test; and/or
(iii) determining that the second dose is the same as the first dose, if the benchmark biomarker test results in an expression level of the thyroid hormone (e.g., FT4) higher than the high exposure threshold, and at the target engagement threshold or lower, as compared to the baseline biomarker test.

In some embodiments, the high exposure threshold for the expression level of the thyroid hormone (e.g., FT4) is about 19% decrease, about 20% decrease, about 21% decrease, about 22% decrease, about 23% decrease, about 24% decrease, about 25% decrease, about 26% decrease, about 27% decrease, about 28% decrease, about 29% decrease, about 30% decrease, about 35% decrease, or about 40% decrease, as compared to the baseline biomarker test.

In some embodiments, the target engagement threshold for the expression level of the thyroid hormone (e.g., FT4) is about 5% decrease, about 6% decrease, about 7% decrease, about 8% decrease, about 9% decrease, about 10% decrease, about 11% decrease, about 12% decrease, about 13% decrease, about 14% decrease, about 15% decrease, or about 16% decrease, as compared to the baseline biomarker test.

In some embodiments, the benchmark biomarker test results in an expression level of the thyroid hormone (e.g., FT4) that is lower, as compared to the baseline biomarker test, by about 19% or greater, about 20% or greater, about 21% or greater, about 22% or greater, about 23% or greater, about 24% or greater, about 25% or greater, about 26% or greater, about 27% or greater, about 28% or greater, about 29% or greater, about 30% or greater, about 35% or greater, or about 40% or greater; and the second dose is determined to be lower than the first dose by about 5 mg, about 10 mg, about 15 mg, about 20 mg, about 25 mg, about 30 mg, about 35 mg, or about 40 mg.

In some embodiments, the benchmark biomarker test results in an expression level of the thyroid hormone (e.g., FT4) that is lower, as compared to the baseline biomarker test, by about 5% or less, about 6% or less, about 7% or less, about 8% or less, about 9% or less, about 10% or less, about 11% or less, about 12% or less, about 13% or less, about 14% or less, about 15% or less, or about 16% or less; and the second dose is determined to be higher than the first dose by about 5 mg, about 10 mg, about 15 mg, about 20 mg, about 25 mg, about 30 mg, about 35 mg, or about 40 mg.

### Second Dose Administrations

In some embodiments, the second dose of Compound A is administered to the subject daily.

In some embodiments, the second dose of Compound A is administered to the subject once daily.

In some embodiments, the second dose of Compound A is administered to the subject two or more times daily.

In some embodiments, the second dose of Compound A is a daily dose of:
about 40±10 mg, about 40±5 mg, about 40±4 mg, about 40±3 mg, about 40±2 mg, or about 40±1 mg (e.g., about 40 mg);
about 60±20 mg, about 60±10 mg, about 60±5 mg, about 60±4 mg, about 60±3 mg, about 60±2 mg, or about 60±1 mg (e.g., about 60 mg);
about 80±20 mg, about 80±10 mg, about 80±5 mg, about 80±4 mg, about 80±3 mg, about 80±2 mg, or about 80±1 mg (e.g., about 80 mg);
about 100±30 mg, about 100±20 mg, about 100±10 mg, about 100±5 mg, about 100±4 mg, about 100±3 mg, about 100±2 mg, or about 100±1 mg (e.g., about 100 mg); or about 120±40 mg, about 120±30 mg, about 120±20 mg, about 120±10 mg, about 120±5 mg, about 120±4 mg, about 120±3 mg, about 120±2 mg, or about 120±1 mg (e.g., about 120 mg).

In some embodiments, the second dose of Compound A is a daily dose of:
about 60±20 mg, about 60±10 mg, about 60±5 mg, about 60±4 mg, about 60±3 mg, about 60±2 mg, or about 60±1 mg (e.g., about 60 mg);
about 80±20 mg, about 80±10 mg, about 80±5 mg, about 80±4 mg, about 80±3 mg, about 80±2 mg, or about 80±1 mg (e.g., about 80 mg); or about 100±30 mg, about 100±20 mg, about 100±10 mg, about 100±5 mg, about 100±4 mg, about 100±3 mg, about 100±2 mg, or about 100±1 mg (e.g., about 100 mg).

In some embodiments, the second dose of Compound A is a daily dose of about 60±20 mg, about 60±10 mg, about 60±5 mg, about 60±4 mg, about 60±3 mg, about 60±2 mg, or about 60±1 mg (e.g., about 60 mg).

In some embodiments, the second dose of Compound A is a daily dose of about 80±20 mg, about 80±10 mg, about 80±5 mg, about 80±4 mg, about 80±3 mg, about 80±2 mg, or about 80±1 mg (e.g., about 80 mg).

In some embodiments, the second dose of Compound A is a daily dose of about 100±30 mg, about 100±20 mg, about 100±10 mg, about 100±5 mg, about 100±4 mg, about 100±3 mg, about 100±2 mg, or about 100±1 mg (e.g., about 100 mg).

In some embodiments, a pharmaceutical composition comprising the second dose of Compound A is administered to the subject. In some embodiments, the pharmaceutical composition is an oral formulation (e.g., a tablet).

It is understood that the second dose of Compound A may be administered to the subject for any period of time, e.g., ranging from one day to 10 years or longer.

In some embodiments, the second dose of Compound A is administered to the subject for about a week or longer, about two weeks or longer, about three weeks or longer, about one month or longer, about two months or longer, about three months or longer, about four months or longer, about five months or longer, about six months or longer, about seven months or longer, about eight months or longer, about nine months or longer, about ten months or longer, about 11 months or longer, or about 12 months or longer.

In some embodiments, the second dose of Compound A is administered to the subject for about one year or longer, for about two years or longer, for about three years or longer, for about four years or longer, for about five years or longer, for about six years or longer, for about seven years or longer, for about eight years or longer, for about nine years or longer, or for about ten years or longer.

In some embodiments, the second dose of Compound A is administered to the subject for about a week, about two weeks, about three weeks, about one month, about two months, about three months, about four months, about five months, about six months, about seven months, about eight months, about nine months, about ten months, about 11 months, or about 12 months.

In some embodiments, the second dose of Compound A is administered to the subject for about one year, for about two years, for about three years, for about four years, for about five years, for about six years, for about seven years, for about eight years, for about nine years, or for about ten years.

It is understood that one or more additional biomarker tests may be performed after the benchmark biomarker test. One or more further doses of Compound A may be determined based on the results of the additional biomarker tests. In some embodiments, the second dose of Compound A is administered to the subject until a further dose of Compound A is determined.

### Additional Tests

In some embodiments, the method further comprises performing one or more baseline tests and/or benchmark tests on one or more metabolic factors. In some embodiments, the method further comprises assessing the results of the one or more baseline tests and/or benchmark tests on one or more metabolic factors

In some embodiments, the metabolic factor is body weight, systolic blood pressure (SBP), diastolic blood pressure (DBP), electrocardiogram (ECG) heart rate, or any combination thereof.

In some embodiments, the method further comprises performing one or more baseline tests and/or benchmark tests on one or more biomarkers, lipids, or lipoproteins, e.g., on enhanced liver fibrosis (ELF), amino-terminal propeptide of type III procollagen (PIIINP), tissue inhibitor of metalloproteinases-1 (TIMP), hyaluronic acid (HA), high-density lipoprotein cholesterol (HDL-C), high-density lipoprotein (HDL), Intermediate-density lipoprotein (IDL), low-density lipoprotein (LDL), very low-density lipoprotein (VLDL), Chylomicron, remnant-like particle (RLP) cholesterol, or VLDL cholesterol.

### Additional Methods Based on Dose-Adjustment Age Thresholds

In some aspects, the present disclosure provides a method of treating or preventing a liver disorder in a subject, comprising:
(i) administering a low-age dose of Compound A to the subject, wherein the subject has an age younger than a dose-adjustment age threshold; and/or
(ii) administering a high-age dose of Compound A to the subject, wherein the subject has an age at a dose-adjustment age threshold or older.

In some aspects, the present disclosure provides a method of treating a liver disorder in a subject, comprising:
(i) administering a low-age dose of Compound A to the subject, wherein the subject has an age younger than a dose-adjustment age threshold; and/or
(ii) administering a high-age dose of Compound A to the subject, wherein the subject has an age at a dose-adjustment age threshold or older.

In some aspects, the present disclosure provides a THR-β agonist for use in treating or preventing a liver disorder in a subject, wherein the treating or preventing comprises:
(i) administering a low-age dose of Compound A to the subject, wherein the subject has an age younger than a dose-adjustment age threshold; and/or
(ii) administering a high-age dose of Compound A to the subject, wherein the subject has an age at a dose-adjustment age threshold or older.

In some aspects, the present disclosure provides a THR-β agonist for use in treating a liver disorder in a subject, wherein the treating or preventing comprises:
(i) administering a low-age dose of Compound A to the subject, wherein the subject has an age younger than a dose-adjustment age threshold; and/or
(ii) administering a high-age dose of Compound A to the subject, wherein the subject has an age at a dose-adjustment age threshold or older.

In some aspects, the present disclosure provides use of Compound A in the manufacture of a medicament for treating or preventing a liver disorder in a subject, wherein the treating comprises:
(i) administering a low-age dose of Compound A to the subject, wherein the subject has an age younger than a dose-adjustment age threshold; and/or
(ii) administering a high-age dose of Compound A to the subject, wherein the subject has an age at a dose-adjustment age threshold or older.

In some aspects, the present disclosure provides use of Compound A in the manufacture of a medicament for treating a liver disorder in a subject, wherein the treating comprises:
(i) administering a low-age dose of Compound A to the subject, wherein the subject has an age younger than a dose-adjustment age threshold; and/or
(ii) administering a high-age dose of Compound A to the subject, wherein the subject has an age at a dose-adjustment age threshold or older.

In some embodiments, the subject has an age younger than a dose-adjustment age threshold, and Compound A is administered to the subject at a low-age dose.

In some embodiments, the subject has an age at a dose-adjustment age threshold or older, and Compound A is administered to the subject at a high-age dose.

### Dose-Adjustment Age Thresholds, Low-Age Doses, and High-Age Doses

In some embodiments, dose-adjustment age threshold is about 50 years old, about 55 years old, about 60 years old, about 65 years old, about 70 years old, about 75 years old, or about 80 years old.

In some embodiments, dose-adjustment age threshold is about 60 years old, about 61 years old, about 62 years old, about 63 years old, about 64 years old, about 65 years old, about 66 years old, about 67 years old, about 68 years old, about 69 years old, or about 70 years old.

In some embodiments, dose-adjustment age threshold is about 65 years old.

As used herein, the term “low-age” refers to an age younger than the dose-adjustment age threshold. Conversely, the term “high-age” refers to an age equal to or older than the dose-adjustment age threshold.

In some embodiments, the low-age dose is a daily dose of:
about 80±20 mg, about 80±10 mg, about 80±5 mg, about 80±4 mg, about 80±3 mg, about 80±2 mg, or about 80±1 mg (e.g., about 80 mg);
about 100±30 mg, about 100±20 mg, about 100±10 mg, about 100±5 mg, about 100±4 mg, about 100±3 mg, about 100±2 mg, or about 100±1 mg (e.g., about 100 mg); or
about 120±40 mg, about 120±30 mg, about 120±20 mg, about 120±10 mg, about 120±5 mg, about 120±4 mg, about 120±3 mg, about 120±2 mg, or about 120±1 mg (e.g., about 120 mg).

In some embodiments, the low-age dose is a daily dose of about 100±30 mg, about 100±20 mg, about 100±10 mg, about 100±5 mg, about 100±4 mg, about 100±3 mg, about 100±2 mg, or about 100±1 mg (e.g., about 100 mg).

In some embodiments, the high-age dose is lower than the low-age dose.

In some embodiments, the high-age dose is lower than the low-age dose by about 5 mg, about 10 mg, about 15 mg, about 20 mg, about 25 mg, about 30 mg, about 35 mg, or about 40 mg.

In some embodiments, the high-age dose is lower than the low-age dose by about 20 mg.

In some embodiments, the high-age dose is a daily dose of:
about 60±20 mg, about 60±10 mg, about 60±5 mg, about 60±4 mg, about 60±3 mg, about 60±2 mg, or about 60±1 mg (e.g., about 60 mg);
about 80±20 mg, about 80±10 mg, about 80±5 mg, about 80±4 mg, about 80±3 mg, about 80±2 mg, or about 80±1 mg (e.g., about 80 mg); or
about 100±30 mg, about 100±20 mg, about 100±10 mg, about 100±5 mg, about 100±4 mg, about 100±3 mg, about 100±2 mg, or about 100±1 mg (e.g., about 100 mg).

In some embodiments, the high-age dose is a daily dose of about 80±20 mg, about 80±10 mg, about 80±5 mg, about 80±4 mg, about 80±3 mg, about 80±2 mg, or about 80±1 mg (e.g., about 80 mg).

In some embodiments, the subject has an age younger than 65 years old, and Compound A is administered to the subject at a daily dose of about 100±30 mg, about 100±20 mg, about 100±10 mg, about 100±5 mg, about 100±4 mg, about 100±3 mg, about 100±2 mg, or about 100±1 mg (e.g., about 100 mg).

In some embodiments, the subject has an age at 65 years old or older, and Compound A is administered to the subject at a daily dose of about 80±20 mg, about 80±10 mg, about 80±5 mg, about 80±4 mg, about 80±3 mg, about 80±2 mg, or about 80±1 mg (e.g., about 80 mg).

### Pharmaceutical Compositions and Kits

In some embodiments, Compound A is present in the pharmaceutical composition at an amount of:
about 40±10 mg, about 40±5 mg, about 40±4 mg, about 40±3 mg, about 40±2 mg, or about 40±1 mg (e.g., about 40 mg);
about 60±20 mg, about 60±10 mg, about 60±5 mg, about 60±4 mg, about 60±3 mg, about 60±2 mg, or about 60±1 mg (e.g., about 60 mg);
about 80±20 mg, about 80±10 mg, about 80±5 mg, about 80±4 mg, about 80±3 mg, about 80±2 mg, or about 80±1 mg (e.g., about 80 mg);
about 100±30 mg, about 100±20 mg, about 100±10 mg, about 100±5 mg, about 100±4 mg, about 100±3 mg, about 100±2 mg, or about 100±1 mg (e.g., about 100 mg); or
about 120±40 mg, about 120±30 mg, about 120±20 mg, about 120±10 mg, about 120±5 mg, about 120±4 mg, about 120±3 mg, about 120±2 mg, or about 120±1 mg (e.g., about 120 mg).

The pharmaceutical compositions of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

The compounds of present disclosure can be formulated for oral administration in forms such as tablets, capsules (each of which includes sustained release or timed release formulations), powder-in-capsules, pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In some embodiments, the active compounds are prepared with pharmaceutically acceptable excipients that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable excipients.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

### Definitions

Unless otherwise defined, all technical and scientific terms used herein have the same meaning as commonly understood by one of ordinary skill in the art to which this disclosure belongs. The terminology used in the description is intended to describe particular embodiments only, and is not intended to limit the scope of the invention.

Where a range of values is provided, it is understood that the range includes both of the endpoints with that range, as well as all intervening values.

The following terms are used to describe the present disclosure. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an ultrapure form” means one ultrapure form or more than one ultrapure form.

The phrase “and/or,” as used herein in the specification and in the claims, should be understood to mean “either or both”. Other elements may optionally be present other than the elements specifically identified by the “and/or” clause. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

Unless explicitly indicated otherwise, the terms “approximately” and “about” are synonymous. In some embodiments, “approximately” and “about” refer to the recited amount, value, dose or duration ±10%, ±8%, ±6%, ±5%, ±4%, ±2%, ±1%, or ±0.5%. In some embodiments, “approximately” and “about” refer to the listed amount, value, dose, or duration ±5%. In some embodiments, “approximately” and “about” refer to the listed amount, value, dose, or duration ±2%. In some embodiments, “approximately” and “about” refer to the listed amount, value, dose, or duration ±1%.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The term “effective” can mean, but is in no way limited to, that amount/dose of the active pharmaceutical ingredient, which, when used in the context of its intended use, effectuates or is sufficient to prevent, inhibit the occurrence, ameliorate, delay or treat (alleviate a symptom to some extent, preferably all) the symptoms of a liver disorder or a lipid disorder in a subject in need of such treatment or receiving such treatment. The term “effective” subsumes all other effective amount or effective concentration terms, e.g., “effective amount/dose,” “pharmaceutically effective amount/dose” or “therapeutically effective amount/dose,” which are otherwise described or used in the present application.

The effective amount depends on the age, weight, gender, previous patient history or family history, type and severity of disease, the composition used, the route of administration, the stage of treatment, the type of mammal being treated, the physical characteristics of the specific mammal under consideration, concurrent medication, and other factors which those skilled in the medical arts will recognize. The exact amount can be ascertainable by one skilled in the art using known techniques in view of clinical data and medical experience (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “salt” is a pharmaceutically acceptable salt and can include acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates. A salt can also be formed between a cation and a negatively charged group on Compound A. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. A salt can also contain a quaternary nitrogen atom.

As used herein, the term “subject” is interchangeable with the term “subject in need thereof”, both of which refer to a subject having a disease or disorder or having an increased risk of developing the disease or disorder. A “subject” includes a mammal. The mammal can be, e.g., a human or appropriate non-human mammal, such as primate, mouse, rat, dog, cat, cow, horse, goat, camel, sheep or a pig. In one embodiment, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

It is to be appreciated that references to “treating” or “treatment” include the alleviation of established symptoms of a condition or disorder. “Treating” or “treatment” of a state, disorder, or condition therefore includes: (1) delaying the appearance of clinical symptoms of the state, disorder, or condition developing in a human that may be afflicted with the state, disorder, or condition but does not yet experience or display clinical or subclinical symptoms of the state, disorder, or condition, (2) inhibiting the state, disorder, or condition, i.e., arresting or reducing the development of the disease or a relapse thereof (in case of maintenance treatment) or at least one clinical or subclinical symptom thereof, or (3) relieving or attenuating the disease, i.e., causing regression of the state, disorder, or condition or at least one of its clinical or subclinical symptoms.

As used herein, the term “preventing” or “prevent” describes reducing or eliminating the onset of the symptoms or complications of a disease, condition, or disorder.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed invention. Based on the present disclosure the skilled artisan can identify and employ other components and methodology useful for practicing the present disclosure.

### EXAMPLES

It is understood that the values shown in the examples below are approximate, and the values are subject to experimental and instrumental variations.

### Example 1. Evaluation of the Safety and Effect of Resmetirom

A 52-week randomized, double-blind, placebo-controlled Phase 3 trial was conducted to evaluate the safety and effect of resmetirom, an oral, once-daily, liver-targeted thyroid hormone receptor-β selective agonist, on multiple biomarkers in adults with nonalcoholic fatty liver disease (NAFLD)/presumed nonalcoholic steatohepatitis (NASH). Patients were randomized to three double-blind arms (100 mg resmetirom, 80 mg resmetirom, or placebo) or one open-label arm (100 mg resmetirom). Common concomitant medications across the 4 arms were antidiabetes drugs (such as glucagon-like peptide-1 receptor agonists (GLP-1 RAs), metformin, pioglitazone, and sodium/glucose cotransporter-2 inhibitors (SGLT2i)) and drugs to manage dyslipidemia (statins).

Minimal reductions from baseline in prohormone FT4 levels were observed in the resmetirom arms. No effects on active hormone free triiodothyronine (FT3) or thyroid-stimulating hormone (TSH) were noted in the resmetirom arms. Sex hormone levels after 52 weeks of resmetirom treatment are shown in Table A. Thyroid hormone levels after 52 weeks of resmetirom treatment are reported in Table B.

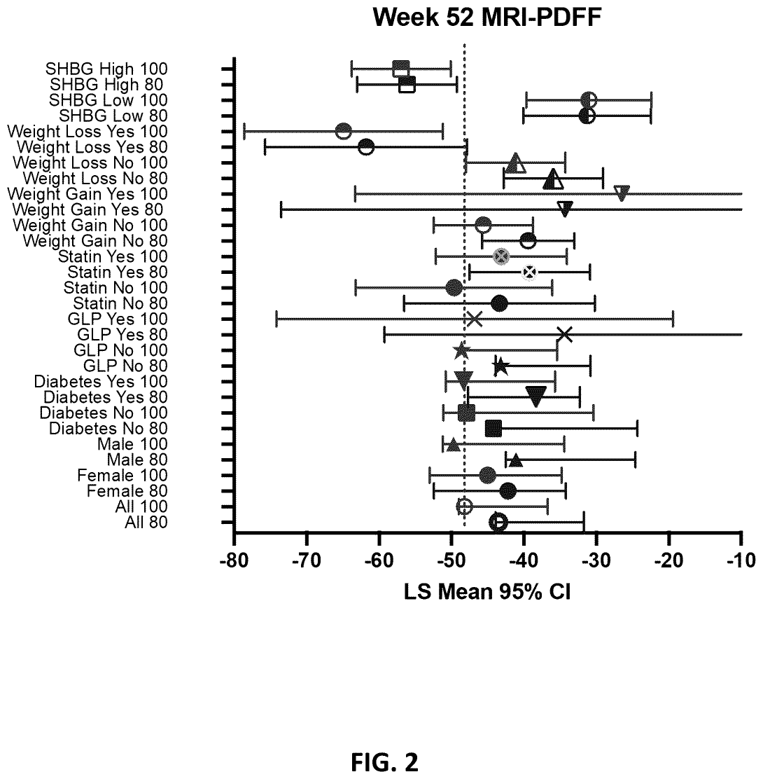

Magnetic resonance imaging-proton density fat fraction (MRI-PDFF) subgroup analyses demonstrated that resmetirom treatment reduced hepatic fat from baseline at Week 52 in all key patient subgroups (FIG. 2). In particular, weight loss ≥5% in combination with resmetirom treatment or high exposure to resmetirom measured by the sex hormone binding globulin (SHBG) response as described previously (defined in this study as ≥120% increase from baseline in SHBG which corresponds to the upper two tertiles of the SHBG response observed with 100 mg resmetirom) was associated with greater reduction in hepatic fat. In contrast, weight gain ≥5% or lower exposure to resmetirom (defined as <120% increase in SHBG) showed lower hepatic fat reduction with resmetirom. In general, 80 mg resmetirom was less effective than 100 mg resmetirom at reducing hepatic fat; however, the effect of 80 mg was similar to 100 mg in females and in patients with ≥120% increase in SHBG.

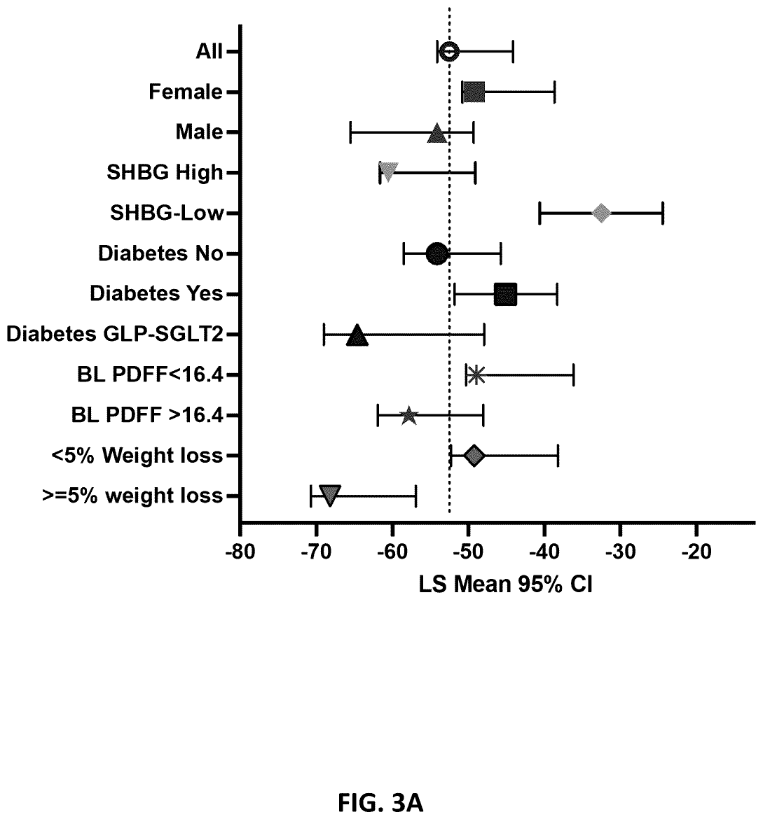

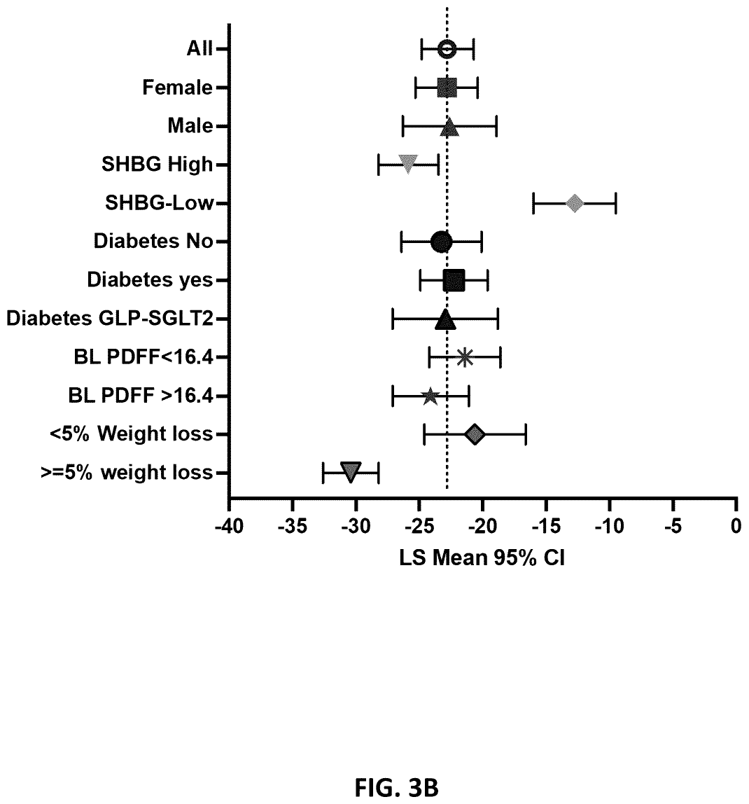

Liver volume was evaluated in the open-label 100 mg resmetirom arm at Week 52. Liver volume was reduced from baseline by a mean of 21% and 23% following 16 and 52 weeks of resmetirom treatment, respectively (FIGS. 3A and 3B). Furthermore, reductions in liver volume were observed in all key patient subgroups of the OL arm. Correcting hepatic fat reduction for reduced liver volume led to an average hepatic fat reduction of 61% in the open-label arm.

TABLE A

| | | | | | | | | Placebo |
|---|---|---|---|---|---|---|---|---|
| Resmetirom 100 mg | | Resmetirom 100 mg DB | | | | | | DB |
| OL (n = 169) | | (n = 314) | | | Resmetirom 80 mg DB (n = 320) | | | (n = 309) |
| LSM CFB | | LSM CFB | LSM | | LSM CFB | LSM | | LSM CFB |
| or % CFB | | or % CFB | Difference | | or % CFB | Difference | | or % CFB |
| (SE) | 95% CI | (SE) | (97.5% CI) | p-value | (SE) | (97.5% CI) | p-value | (SE) |

Estradiol, ng/L (female)

| n | 105 | | 145 | | | 138 | | | 127 |
|---|---|---|---|---|---|---|---|---|---|
| Baseline | 35.7 (58.7) | | 41.9 (66.5) | | | 37.4 (70.0) | | | 38.6 |
| mean (SD) | | | | | | | | | (59.6) |
| Week 52 | 11.0 | −6.3 to | 7.5 | 9.5 | 0.26 | 5.0 | 7.1 | 0.41 | −2.1 |
| CFB (SE) | (8.8) | 28.4 | (8.2) | (−9.5 | | (8.3) | (−12.2 | | (8.4) |
| | | | | to 28.6) | | | to 26.3) | | |

Estradiol, ng/L (male)

| n | 46 | | 118 | | | 111 | | | 129 |
|---|---|---|---|---|---|---|---|---|---|
| Baseline | 28.4 (10.7) | | 34.3 (14.2) | | | 35.9 (18.5) | | | 32.8 |
| mean (SD) | | | | | | | | | (14.9) |
| Week 52 | 11.7 | 7.4 to | 8.9 | 10.4 | <0.0001 | 3.3 | 4.8 | 0.0037 | −1.5 |
| CFB (SE) | (2.2) | 15.9 | (1.5) | (6.8 to | | (1.5) | (1.1 to | | (1.5) |
| | | | | 14.1) | | | 8.6) | | |

FSH, IU/L (female)

| n | 105 | | 145 | | | 138 | | | 126 |
|---|---|---|---|---|---|---|---|---|---|
| Baseline | 36.4 (25.7) | | 40.0 (27.5) | | | 41.5 (25.7) | | | 38.0 |
| mean (SD) | | | | | | | | | (25.2) |
| Week 52 | 1.3 | −1.0 | 1.6 | −0.4 | 0.72 | 0.9 | −1.1 | 0.35 | 2.0 |
| CFB (SE) | (1.2) | to 3.6 | (1.1) | (−2.9 | | (1.1) | (−3.6 | | (1.1) |
| | | | | to 2.1) | | | to 1.5) | | |

FSH, IU/L (male)

| n | 46 | | 118 | | | 111 | | | 129 |
|---|---|---|---|---|---|---|---|---|---|
| Baseline | 6.3 (3.3) | | 6.3 (5.2) | | | 6.6 (7.1) | | | 6.5 |
| mean (SD) | | | | | | | | | (5.9) |
| Week 52 | 0.6 | −0.2 | 0.6 | 0.5 | 0.12 | 0.3 | 0.2 | 0.58 | 0.1 |
| CFB (SE) | (0.4) | to 1.3 | (0.3) | (−0.2 | | (0.3) | (−0.5 | | (0.3) |
| | | | | to 1.1) | | | to 0.8) | | |

LH, IU/L (female)

| n | 105 | | 143 | | | 138 | | | 126 |
|---|---|---|---|---|---|---|---|---|---|
| Baseline | 21.1 (12.8) | | 23.6 (13.6) | | | 24.6 (14.1) | | | 22.1 |
| mean (SD) | | | | | | | | | (12.9) |
| Week 52 | −0.0 | −1.9 | 1.2 | −0.5 | 0.61 | 0.8 | −0.9 | 0.34 | 1.7 |
| CFB (SE) | (0.9) | to 1.8 | (0.9) | (−2.5 | | (0.9) | (−2.9 | | (0.9) |
| | | | | to 1.6) | | | to 1.2) | | |

LH, IU/L (male)

| n | 46 | | 118 | | | 111 | | | 129 |
|---|---|---|---|---|---|---|---|---|---|
| Baseline | 5.6 (3.0) | | 5.8 (3.0) | | | 5.8 (5.1) | | | 6.0 |
| mean (SD) | | | | | | | | | (3.5) |
| Week 52 | 0.3 | −0.5 | 0.9 | 0.8 | 0.01 | 0.4 | 0.4 | 0.28 | 0.1 |
| CFB (SE) | (0.4) | to 1.2 | (0.3) | (0.1 to | | (0.3) | (−0.4 | | (0.3) |
| | | | | 1.6) | | | to 1.1) | | |

Testosterone, ug/L (female)

| n | 105 | | 145 | | | 138 | | | 127 |
|---|---|---|---|---|---|---|---|---|---|
| Baseline | 0.13 (0.14) | | 0.17 (0.25) | | | 0.18 (0.30) | | | 0.17 |
| mean (SD) | | | | | | | | | (0.19) |
| Week 52 | 0.1 | 0.0 to | 0.1 | 0.1 | <0.0001 | 0.1 | 0.1 | <0.0001 | 0.0 |
| CFB (SE) | (0.0) | 0.1 | (0.0) | (0.1 to | | (0.0) | (0.1 to | | (0.0) |
| | | | | 0.2) | | | 0.2) | | |

Testosterone, ug/L (male)

| n | 46 | | 118 | | | 111 | | | 129 |
|---|---|---|---|---|---|---|---|---|---|
| Baseline | 3.7 (1.6) | | 3.6 (1.5) | | | 3.8 (2.5) | | | 3.6 |
| mean (SD) | | | | | | | | | (1.7) |
| Week 52 | 2.8 | 2.1 to | 2.0 | 2.1 | <0.0001 | 1.4 | 1.5 | <0.0001 | −0.1 |
| CFB (SE) | (0.4) | 3.5 | (0.3) | (1.4 to | | (0.3) | (0.9 to | | (0.3) |
| | | | | 2.7) | | | 2.2) | | |

Free testosterone, ug/L (female)

| n | 75 | | 112 | | | 96 | | | 91 |
|---|---|---|---|---|---|---|---|---|---|
| Baseline | 0.0031 (0.0029) | | 0.0039 (0.0059) | | | 0.0038 (0.0048) | | | 0.0035 |
| mean (SD) | | | | | | | | | (0.0028) |
| Week 52 | −0.0006 | −0.0015 | −0.0003 | −0.0006 | 0.12 | 0.0004 | 0.0002 | 0.61 | 0.0002 |
| CFB (SE) | (0.0004) | to 0.0003 | (0.0004) | (−0.0014 | | (0.0004) | (−0.0007 | | (0.0004) |
| | | | | to 0.0003) | | | to 0.0011) | | |

Free testosterone, ug/L (male)

| n | 45 | | 115 | | | 108 | | | 127 |
|---|---|---|---|---|---|---|---|---|---|
| Baseline | 0.0690 (0.0218) | | 0.0718 (0.0315) | | | 0.0758 (0.0498) | | | 0.0713 |
| mean (SD) | | | | | | | | | (0.0370) |
| Week 52 | 0.024 | −0.0081 | −0.0009 | 0.0029 | 0.48 | 0.0077 | 0.0115 | 0.00054 | −0.0038 |
| CFB (SE) | (0.0054) | to 0.0130 | (0.0038) | (−0.0061 | | (0.0037) | (0.0023 to | | (0.0036) |
| | | | | to 0.0120) | | | 0.0208) | | |

SHBG, nmol/L

| n | 151 | | 263 | | | 248 | | | 255 |
|---|---|---|---|---|---|---|---|---|---|
| Baseline | 37.7 (24.4) | | 35.8 (19.8) | | | 35.8 (20.7) | | | 35.1 |
| mean (SD) | | | | | | | | | (18.7) |
| Week | 182.6 | 157.0 to | 161.1 | 157.5 | <0.0001 | 99.6 | 96.0 | <0.0001 | 3.6 |
| 52 % CFB | (13.0) | 208.1 | (11.0) | (131.5 to | | (10.9) | (69.6 to | | (10.9) |
| (SE) | | | | 183.4) | | | 122.4) | | |
| Week 52 | 60.3 | 51.7 to | 53.0 | 52.4 | <0.0001 | 32.7 | 32.1 | <0.0001 | 0.6 |
| CFB (SE) | (4.4) | 68.9 | (3.7) | (43.6 to | | (3.7) | (23.2 to | | (3.7) |
| | | | | 61.2) | | | 41.0) | | |

SHBG, nmol/L (female)

| n | 105 | | 145 | | | 137 | | | 126 |
|---|---|---|---|---|---|---|---|---|---|
| Baseline | 38.7 (26.0) | | 38.9 (22.7) | | | 40.7 (24.1) | | | 36.8 |
| mean (SD) | | | | | | | | | (20.5) |
| Week | 199.3 | 160.8 to | 197.6 | 184.0 | <0.0001 | 133.0 | 119.5 | <0.0001 | 13.5 |
| 52 % CFB | (19.6) | 237.8 | (18.2) | (141.5 to | | (18.4) | (76.3 to | | (18.7) |
| (SE) | | | | 226.5) | | | 162.6) | | |
| Week 52 | 68.6 | 55.5 to | 65.3 | 61.8 | <0.0001 | 43.3 | 39.9 | <0.0001 | 3.5 |
| CFB (SE) | (6.6) | 81.4 | (6.1) | (47.5 to | | (6.2) | (25.4 to | | (6.3) |
| | | | | 76.1) | | | 54.4) | | |

SHBG, nmol/L (male)

| n | 46 | | 118 | | | 111 | | | 129 |
|---|---|---|---|---|---|---|---|---|---|
| Baseline | 35.5 (20.2) | | 32.1 (14.8) | | | 29.8 (13.4) | | | 33.5 |
| mean (SD) | | | | | | | | | (16.6) |
| Week | 156.0 | 126.9 to | 122.9 | 125.5 | <0.0001 | 67.1 | 69.7 | <0.0001 | −2.6 |
| 52 % CFB | (14.8) | 185.1 | (10.5) | (100.4 to | | (10.2) | (44.1 to | | (10.0) |
| (SE) | | | | 150.5) | | | 95.2) | | |
| Week 52 | 44.7 | 34.8 to | 39.2 | 41.7 (3.8) | <0.0001 | 21.9 | 24.5 (3.9) | <0.0001 | −2.5 |
| CFB (SE) | (5.0) | 54.6 | (3.6) | (33.2 to | | (3.5) | (15.8 to | | (3.4) |
| | | | | 50.2) | | | 33.2) | | |

Sex hormone binding globulin (SHBG) reported as both change from baseline (CFB) and percent change from baseline (% CFB) at Week 52. All other sex hormones reported as change from baseline (CFB) at Week 52. CFB, change from baseline; CI, confidence interval; DB, double-blind; FSH, follicle-stimulating hormone; LH, luteinizing hormone; LSM, least squares mean; OL, open-label; SE, standard error; SHBG, sex hormone binding globulin.

TABLE B

| | | | | | | | | Placebo |
|---|---|---|---|---|---|---|---|---|
| Resmetirom 100 mg | | Resmetirom 100 mg DB | | | | | | DB |
| OL (n = 169) | | (n = 314) | | | Resmetirom 80 mg DB (n = 320) | | | (n = 309) |
| LSM CFB | | LSM CFB | LSM | | LSM CFB | LSM | | LSM CFB |
| or % CFB | | or % CFB | Difference | | or % CFB | Difference | | or % CFB |
| (SE) | 95% CI | (SE) | (97.5% CI) | p-value | (SE) | (97.5% CI) | p-value | (SE) |

FT3, ng/L

| n | 151 | | 263 | | | 250 | | | 257 |
|---|---|---|---|---|---|---|---|---|---|
| Baseline | 2.8 (0.5) | | 2.9 (0.4) | | | 2.9 (0.5) | | | 3.0 |
| mean (SD) | | | | | | | | | (0.4) |
| Week 52 | −0.2 | −0.2 | −0.1 | 0.0 | 0.87 | −0.0 | 0.1 | 0.24 | −0.1 |
| CFB | (0.0) | to −0.0 | (0.0) | (−0.1 | | (0.0) | (−0.0 | | (0.0) |
| | | | | to 0.1) | | | to 0.1) | | |

FT3, ng/L (not on thyroxine)

| n | 81 | | 234 | | | 217 | | | 231 |
|---|---|---|---|---|---|---|---|---|---|
| Baseline | 3.0 (0.4) | | 3.0 (0.4) | | | 2.9 (0.4) | | | 3.0 |
| mean (SD) | | | | | | | | | (0.4) |
| Week 52 | −0.1 | −0.2 | −0.1 | 0.0 | 0.84 | −0.1 | 0.1 | 0.19 | −0.1 |
| CFB | (0.1) | to 0.0 | (0.0) | (−0.1 | | (0.0) | (−0.0 | | (0.0) |
| | | | | to 0.1) | | | to 0.2) | | |

FT3, ng/L (thyroxine-treated)

| n | 70 | | 29 | | | 33 | | | 26 |
|---|---|---|---|---|---|---|---|---|---|
| Baseline | 2.7 (0.6) | | 2.6 (0.4) | | | 2.9 (0.7) | | | 2.7 |
| mean (SD) | | | | | | | | | (0.4) |
| Week 52 | −0.1 | −0.3 to | −0.0 | −0.0 | 0.98 | 0.0 | 0.0 | 0.80 | −0.0 |
| CFB | (0.1) | 0.0 | (0.1) | (−0.3 | | (0.1) | (−0.2 | | (0.1) |
| | | | | to 0.3) | | | to 0.3) | | |

FT4, ng/dL

| n | 151 | | 263 | | | 250 | | | 257 |
|---|---|---|---|---|---|---|---|---|---|
| Baseline | 1.2 (0.2) | | 1.1 (0.2) | | | 1.1 (0.2) | | | 1.1 |
| mean (SD) | | | | | | | | | (0.2) |
| Week | −16.1 | −19.5 | −14.9 | −16.1 | <0.0001 | −9.6 | −10.8 | <0.0001 | 1.2 |
| 52 % CFB | (1.8) | to −12.7 | (1.5) | (−19.5 | | (1.4) | (−14.3 | | (1.4) |
| | | | | to −12.6) | | | to −7.3) | | |
| Week 52 | −0.2 | −0.2 | −0.2 | −0.2 | <0.0001 | −0.1 | −0.1 | <0.0001 | 0.0 |
| CFB | (0.2) | to −0.2 | (0.0) | (−0.2 | | (0.0) | (−0.2 | | (0.0) |
| | | | | to −0.1) | | | to −0.1) | | |

FT4, ng/dL (not on thyroxine)

| n | 81 | | 234 | | | 217 | | | 231 |
|---|---|---|---|---|---|---|---|---|---|
| Baseline | 1.1 (0.2) | | 1.1 (0.2) | | | 1.1 (0.2) | | | 1.1 |
| mean (SD) | | | | | | | | | (0.2) |
| Week | −18.9 | −23.3 | −14.3 | −15.4 | <0.0001 | −9.9 | −11.0 | <0.0001 | 1.1 |
| 52 % CFB | (2.2) | to −14.5 | (1.6) | (−19.0 | | (1.5) | (−14.7 | | (1.6) |
| | | | | to −11.7) | | | to −7.3) | | |
| Week 52 | −0.2 | −0.3 | −0.2 | −0.2 | <0.0001 | −0.1 | −0.1 | <0.0001 | 0.0 |
| CFB | (0.0) | to −0.2 | (0.0) | (−0.2 | | (0.0) | (−0.2 | | (0.0) |
| | | | | to −0.1) | | | to −0.1) | | |

FT4, ng/dL (thyroxine-treated)

| n | 70 | | 29 | | | 33 | | | 26 |
|---|---|---|---|---|---|---|---|---|---|
| Baseline | 1.3 (0.2) | | 1.1 (0.2) | | | 1.3 (0.3) | | | 1.2 |
| mean (SD) | | | | | | | | | (0.2) |
| Week | −14.8 | −20.8 | −19.2 | −23.0 | <0.01 | −6.4 | −10.2 | 0.0251 | 3.8 |
| 52 % CFB | (3.0) | to −8.8 | (3.8) | (−33.5 | | (3.9) | (−20.3 | | (3.9) |
| | | | | to −12.5) | | | to 0.0) | | |
| Week 52 | −0.2 | −0.3 | −0.2 | −0.3 | <0.0001 | −0.1 | −0.1 | 0.0118 | 0.0 |
| CFB | (0.0) | to −0.1 | (0.0) | (−0.4 | | (0.0) | (−0.3 | | (0.0) |
| | | | | to −0.1) | | | to −0.0) | | |

rT3, ng/dL

| n | 152 | | 265 | | | 250 | | | 257 |
|---|---|---|---|---|---|---|---|---|---|
| Baseline | 18.1 (5.5) | | 16.3 (4.6) | | | 17.8 (5.1) | | | 16.8 |
| mean (SD) | | | | | | | | | (4.6) |
| Week 52 | −3.3 | −4.1 | −3.2 | −3.9 | <0.0001 | −2.7 | −3.4 | <0.0001 | 0.7 |
| CFB | (0.4) | to −2.6 | (0.3) | (−4.7 | | (0.3) | (−4.2 | | (0.3) |
| | | | | to −3.2) | | | to −2.7) | | |

rT3, ng/dL (not on thyroxine)

| n | 82 | | 236 | | | 217 | | | 231 |
|---|---|---|---|---|---|---|---|---|---|
| Baseline | 16.5 (5.1) | | 16.1 (4.2) | | | 17.5 (4.9) | | | 16.5 |
| mean (SD) | | | | | | | | | (4.4) |
| Week 52 | −3.5 | −4.4 | −2.9 | −3.8 | <0.0001 | −2.5 | −3.3 | <0.0001 | 0.9 |
| CFB | (0.5) | to −2.6 | (0.3) | (−4.5 | | (0.3) | (−4.1 | | (0.3) |
| | | | | to −3.0) | | | to −2.6) | | |

rT3, ng/dL (thyroxine-treated)

| n | 70 | | 29 | | | 33 | | | 26 |
|---|---|---|---|---|---|---|---|---|---|
| Baseline | 20.1 (5.3) | | 18.1 (6.8) | | | 19.6 (5.6) | | | 18.6 |
| mean (SD) | | | | | | | | | (5.5) |
| Week 52 | −3.9 | −5.5 | −4.9 | −5.5 | <0.0001 | −3.8 | −4.4 | 0.0004 | 0.6 |
| CFB | (0.8) | to −2.2 | (1.0) | (−8.3 | | (1.0) | (−7.1 | | (1.0) |
| | | | | to −2.7) | | | to −1.7) | | |

FT3/rT3

| n | 151 | | 263 | | | 249 | | | 256 |
|---|---|---|---|---|---|---|---|---|---|
| Baseline | 0.17 (0.07) | | 0.19 (0.06) | | | 0.18 (0.06) | | | 0.19 |
| mean (SD) | | | | | | | | | (0.06) |
| Week 52 | 0.04 | 0.03 to | 0.04 | 0.05 | <0.0001 | 0.04 | 0.05 | <0.0001 | −0.01 |
| CFB | (0.01) | 0.05 | (0.01) | (0.04 to | | (0.01) | (0.03 to | | (0.001) |
| | | | | 0.07) | | | 0.06) | | |

FT3/rT3 (not on thyroxine)

| n | 81 | | 234 | | | 216 | | | 230 |
|---|---|---|---|---|---|---|---|---|---|
| Baseline | 0.20 (0.006) | | 0.20 (0.006) | | | 0.18 (0.006) | | | 0.19 |
| mean (SD) | | | | | | | | | (0.006) |
| Week 52 | 0.05 | 0.03 to | 0.04 | 0.05 | <0.0001 | 0.04 | 0.05 | <0.0001 | −0.01 |
| CFB | (0.01) | 0.07 | (0.01) | (0.04 to | | (0.01) | (0.04 to | | (0.01) |
| | | | | 0.07) | | | 0.006) | | |

FT3/rT3 (thyroxine-treated)

| n | 70 | | 29 | | | 33 | | | 26 |
|---|---|---|---|---|---|---|---|---|---|
| Baseline | 0.14 (0.07) | | 0.16 (0.05) | | | 0.16 (0.07) | | | 0.16 |
| mean (SD) | | | | | | | | | (0.06) |
| Week 52 | 0.03 | 0.01 to | 0.04 | 0.04 | <0.0023 | 0.03 | 0.03 | 0.0136 | 0.00 |
| CFB | (0.01) | 0.05 | (0.01) | (0.01 to | | (0.01) | (0.00 to | | (0.01) |
| | | | | 0.07) | | | 0.06) | | |

TSH, mIU/L

| n | 151 | | 263 | | | 250 | | | 256 |
|---|---|---|---|---|---|---|---|---|---|
| Baseline | 2.3 (1.8) | | 2.3 (1.9) | | | 2.0 (1.1) | | | 2.2 |
| mean (SD) | | | | | | | | | (1.1) |
| Week 52 | 0.2 | −0.4 | −0.4 | −0.1 | 0.81 | −0.4 | −0.0 | 0.88 | −0.3 |
| CFB | (0.3) | to 0.7 | (0.2) | (−0.6 | | (0.2) | (−0.6 | | (0.2) |
| | | | | to 0.5) | | | to 0.5) | | |

TSH, mIU/L (not on thyroxine)

| n | 81 | | 234 | | | 217 | | | 230 |
|---|---|---|---|---|---|---|---|---|---|
| Baseline | 2.2 (1.3) | | 2.2 (1.3) | | | 2.0 (1.1) | | | 2.2 |
| mean (SD) | | | | | | | | | (1.0) |
| Week 52 | −0.2 | −0.4 | −0.3 | −0.1 | 0.33 | −0.3 | 0.0 | 0.96 | −0.3 |
| CFB | (0.1) | to −0.0 | (0.1) | (−0.2 | | (0.1) | (−0.1 | | (0.1) |
| | | | | to 0.1) | | | to 0.2) | | |

TSH, mIU/L (thyroxine-treated)

| n | 70 | | 29 | | | 33 | | | 26 |
|---|---|---|---|---|---|---|---|---|---|
| Baseline | 2.4 (2.3) | | 3.0 (4.4) | | | 1.6 (1.2) | | | 2.2 |
| mean (SD) | | | | | | | | | (1.6) |
| Week 52 | 0.4 | −1.9 | −0.6 | 0.0 | 0.99 | −0.9 | −0.3 | 0.89 | −0.6 |
| CFB | (1.2) | to 2.7 | (1.5) | (−4.0 | | (1.5) | (−4.2 | | (1.5) |
| | | | | to 4.1) | | | to 3.7) | | |

TBG, mg/L

| n | 152 | | 263 | | | 247 | | | 256 |
|---|---|---|---|---|---|---|---|---|---|
| Baseline | 23.9 (6.2) | | 21.6 (6.5) | | | 21.6 (6.2) | | | 21.2 |
| mean (SD) | | | | | | | | | (5.5) |
| Week 52 | 0.1 | −0.9 | 1.1 | −0.5 | 0.24 | 0.6 | −1.1 | 0.88 | 1.7 |
| CFB | (0.5) | to 0.9 | (0.4) | (−1.5 | | (0.4) | (−2.0 | | (0.4) |
| | | | | to 0.5) | | | to −0.1) | | |

TBG, mg/L (not on thyroxine)

| n | 82 | | 234 | | | 214 | | | 230 |
|---|---|---|---|---|---|---|---|---|---|
| Baseline | 22.8 (4.7) | | 21.3 (6.4) | | | 21.5 (6.0) | | | 21.3 |
| mean (SD) | | | | | | | | | (5.3) |
| Week 52 | 0.7 | −0.6 to | 1.4 | −0.3 | 0.56 | −0.5 | −1.2 | 0.01 | 1.7 |
| CFB | (0.6) | 1.9 | (0.5) | (−1.3 to | | (0.4) | (−2.2 | | (0.4) |
| | | | | 0.8) | | | to −0.1) | | |

TBG, mg/L (thyroxine-treated)

| n | 70 | | 29 | | | 33 | | | 26 |
|---|---|---|---|---|---|---|---|---|---|
| Baseline | 25.3 (7.4) | | 24.5 (6.0) | | | 22.0 (7.6) | | | 20.3 |
| mean (SD) | | | | | | | | | (7.2) |
| Week 52 | −1.0 | −2.6 | −0.2 | −2.4 | 0.06 | 1.5 | −0.6 | 0.61 | 2.1 |
| CFB | (0.8) | to 0.6 | (1.0) | (−5.2 | | (1.0) | (−3.3 | | (1.1) |
| | | | | to 0.5) | | | to 2.1) | | |

Free thyroxine (FT4) reported as both change from baseline (CFB) and percent change from baseline (% CFB) at Week 52. All other thyroid hormones reported as change from baseline (CFB) at Week 52. CFB, change from baseline; CI, confidence interval; DB, double-blind; LSM, least squares mean; OL, open-label; RT3, reverse triiodothyronine; SE, standard error; FT3, free triiodothyronine; FT4, free thyroxine; TBG, thyroxine binding globulin; TSH, thyroid-stimulating hormone.

### Example 2. A Randomized Controlled Study of Resmetirom in Nonalcoholic Steatohepatitis

A randomized, double-blind, placebo-controlled Phase 3 trial was conducted. Patients were randomly assigned to receive resmetirom 80 mg, resmetirom 100 mg, or placebo administered orally once daily. Screening biopsy results were used as baseline for histologic variables and a second biopsy was performed at Week 52.

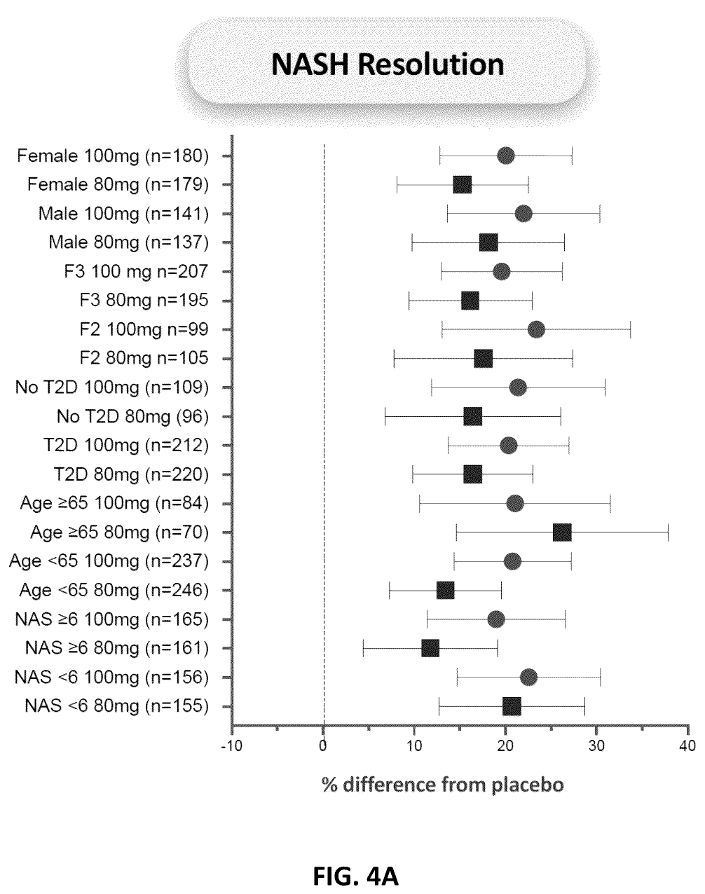

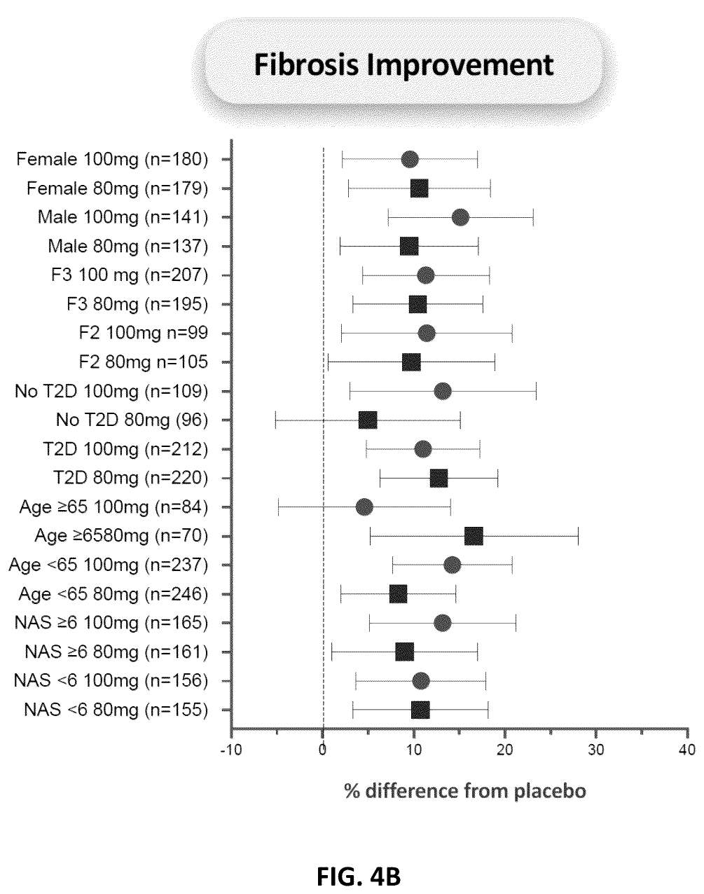

Subgroup analyses of the primary endpoints revealed consistent results across subgroups (based on baseline fibrosis stage, baseline NAS, type 2 diabetes status, age, and sex) with significantly more resmetirom-treated patients achieving either NASH resolution or fibrosis improvement compared with placebo-treated patients. The 100 mg resmetirom group generally outperformed the 80 mg group except in patients ≥65 years (FIGS. 4A and 4B). Patients with ≥30% liver fat reduction or increase in the exposure biomarker SHBG by ≥120% had a greater response at both endpoints. Change in sex hormones from baseline are reported and were unchanged, except for SHBG increases (Table C). Thyroid hormone indices were not clinically impacted by resmetirom treatment (Table D). Resmetirom treatment reduced prohormone T4 as reflective by free thyroxine (FT4) by approximately 16-19% in the resmetirom groups but had no effect on thyroid-stimulating hormone (TSH) or the active thyroid hormone, free triiodothyronine (FT3), independent of thyroxine replacement (Table D).

TABLE C

| | | | | | | |
|---|---|---|---|---|---|---|
| LS | LS | | LS Mean | | LS Mean | |
| Mean % CFB | Mean % CFB | LS | Difference | | Difference | |
| or CFB (SE) | or CFB (SE) | Mean % CFB | Resmetirom | | Resmetirom | |
| Resmetirom | Resmetirom | or CFB (SE) | 80 mg from | | 100 mg | |
| 80 mg | 100 mg | Placebo | PBO | | from PBO | |
| (n = 322) | (n = 323) | (n = 321) | (95% CI) | p-value | (95% CI) | p-value |

Estradiol, ng/L (female)

| | | | | | | | |
|---|---|---|---|---|---|---|---|
| no. | 160 | 147 | 155 | | | | |
| Baseline | 28.6 (37.0) | 32.1 (56.3) | 32.8 (65.9) | | | | |
| mean (SD) | | | | | | | |
| Week 52 | 17.7 (8.0) | 30.6 (8.3) | 1.8 (8.0) | 15.9 | 0.0699 | 28.8 | 0.0013 |
| CFB (SE) | | | | (−1.3, 33.1) | | (11.3, 46.3) | |

Estradiol, ng/L (male)

| | | | | | | | |
|---|---|---|---|---|---|---|---|
| no. | 118 | 118 | 128 | | | | |
| Baseline | 28.0 (11.6) | 27.6 (10.9) | 29.3 (12.1) | | | | |
| mean (SD) | | | | | | | |
| Week 52 | 8.9 (1.3) | 11.0 (1.3) | −0.15 (1.2) | 9.0 | <0.0001 | 11.2 | <0.0001 |
| CFB (SE) | | | | (6.3, 11.8) | | (8.4, 13.9) | |

FSH, mIU/mL (female)

| | | | | | | | |
|---|---|---|---|---|---|---|---|
| no. | 160 | 148 | 155 | | | | |
| Baseline | 39.2 (25.7) | 39.3 (22.6) | 39.8 (23.2) | | | | |
| mean (SD) | | | | | | | |
| Week 52 | −0.54 (0.89) | 0.63 (0.92) | −1.3 (0.89) | 0.79 | 0.4173 | 2.0 | 0.0478 |
| CFB (SE) | | | | (−1.1, 2.7) | | (0.02, 3.9) | |

FSH, mIU/mL (male)

| | | | | | | | |
|---|---|---|---|---|---|---|---|
| no. | 118 | 119 | 128 | | | | |
| Baseline | 8.1 (7.7) | 7.8 (9.7) | 7.2 (6.5) | | | | |
| mean (SD) | | | | | | | |
| Week 52 | 1.1 (0.24) | 1.7 (0.24) | 0.01 (0.22) | 1.1 | <0.0001 | 1.7 | <0.0001 |
| CFB (SE) | | | | (0.57, 1.6) | | (1.1, 2.2) | |

LH, mIU/mL (female)

| | | | | | | | |
|---|---|---|---|---|---|---|---|
| no. | 160 | 148 | 155 | | | | |
| Baseline | 23.5 (14.1) | 24.2 (13.2) | 23.3 (12.0) | | | | |
| mean (SD) | | | | | | | |
| Week 52 | −0.93 (0.69) | 0.80 (0.72) | −0.60 (0.70) | −0.33 | 0.6638 | 1.4 | 0.0688 |
| CFB (SE) | | | | (−1.8, 1.2) | | (−0.11, 2.9) | |

LH, mIU/mL (male)

| | | | | | | | |
|---|---|---|---|---|---|---|---|
| no. | 118 | 119 | 128 | | | | |
| Baseline | 6.3 (4.1) | 6.0 (4.6) | 6.1 (4.0) | | | | |
| mean (SD) | | | | | | | |
| Week 52 | 1.7 (0.30) | 1.9 (0.30) | −0.10 (0.28) | 1.8 | <0.0001 | 2.0 | <0.0001 |
| CFB (SE) | | | | (1.1, 2.4) | | (1.4, 2.7) | |

Testosterone, ug/L (female)

| | | | | | | | |
|---|---|---|---|---|---|---|---|
| no. | 160 | 147 | 156 | | | | |
| Baseline | 0.2 (0.17) | 0.2 (0.16) | 0.1 (0.24) | | | | |
| mean (SD) | | | | | | | |
| Week 52 | 0.15 (0.019) | 0.19 (0.020) | 0 (0.019) | 0.15 | <0.0001 | 0.19 | <0.0001 |
| CFB (SE) | | | | (0.10, 0.19) | | (0.14, 0.23) | |

Testosterone, ug/L (male)

| | | | | | | | |
|---|---|---|---|---|---|---|---|
| no. | 118 | 118 | 128 | | | | |
| Baseline | 3.5 (1.6) | 3.7 (2.0) | 3.3 (1.5) | | | | |
| mean (SD) | | | | | | | |
| Week 52 | 2.6 (0.26) | 3.5 (0.25) | 0.44 (0.24) | 2.2 | <0.0001 | 3.0 | <0.0001 |
| CFB (SE) | | | | (1.6, 2.8) | | (2.5, 3.6) | |

Free testosterone, nmol/L (female)

| | | | | | | | |
|---|---|---|---|---|---|---|---|
| no. | 121 | 110 | 106 | | | | |
| Baseline | 0 (0.01) | 0 (0.01) | 0 (0.01) | | | | |
| mean (SD) | | | | | | | |
| Week 52 | 0 (0.001) | 0 (0.001) | 0 (0.001) | 0 | 0.5438 | 0 | 0.6502 |
| CFB (SE) | | | | | | | |

Free testosterone, nmol/L (male)

| | | | | | | | |
|---|---|---|---|---|---|---|---|
| no. | 116 | 108 | 127 | | | | |
| Baseline | 0.2 (0.09) | 0.2 (0.11) | 0.2 (0.07) | | | | |
| mean (SD) | | | | | | | |
| Week 52 | 0.04 (0.009) | 0.03 (0.009) | 0.02 (0.008) | 0.02 | 0.0686 | 0.01 | 0.3513 |
| CFB (SE) | | | | (0, 0.04) | | (−0.01, 0.03) | |

SHBG, nmol/L

| | | | | | | | |
|---|---|---|---|---|---|---|---|
| no. | 275 | 262 | 283 | | | | |
| Baseline | 48.8 (56.7) | 45.6 (37.9) | 47.2 (43.7) | | | | |
| mean (SD) | | | | | | | |
| Week | 157.5 (10.2) | 217.4 (10.3) | 9.0 (9.8) | 148.5 | <0.0001 | 208.4 | <0.0001 |
| 52 % CFB | | | | (126.6, 170.4) | | (186.3, 230.5) | |
| (SE) | | | | | | | |
| Week 52 | 60.2 (4.0) | 80.7 (4.1) | 1.3 (3.9) | 58.9 | <0.0001 | 79.4 | <0.0001 |
| CFB (SE) | | | | (50.3, 67.5) | | (70.7, 88.1) | |

SHBG, nmol/L (female)

| | | | | | | | |
|---|---|---|---|---|---|---|---|
| no. | 159 | 145 | 155 | | | | |
| Baseline | 58.2 (71.7) | 48.9 (44.5) | 55.7 (54.6) | | | | |
| mean (SD) | | | | | | | |
| Week | 193.0 (15.3) | 251.8 (16.0) | 15.6 (15.5) | 177.4 | <0.0001 | 236.2 | <0.0001 |
| 52 % CFB | | | | (144.3, 210.5) | | (202.3, 270.0) | |
| (SE) | | | | | | | |
| Week 52 | 74.1 (6.0) | 94.0 (6.3) | 0.82 (6.1) | 73.3 | <0.0001 | 93.2 | <0.0001 |
| CFB (SE) | | | | (60.3, 86.2) | | (79.9, 106.5) | |

SHBG, nmol/L (male)

| | | | | | | | |
|---|---|---|---|---|---|---|---|
| no. | 116 | 117 | 128 | | | | |
| Baseline | 36.0 (17.2) | 41.4 (27.1) | 37.0 (20.8) | | | | |
| mean (SD) | | | | | | | |
| Week | 108.0 (11.2) | 174.0 (10.9) | 0.74 (10.1) | 107.3 | <0.0001 | 173.3 | <0.0001 |
| 52 % CFB | | | | (83.4, 131.2) | | (149.4, 197.2) | |
| (SE) | | | | | | | |
| Week 52 | 41.9 (4.2) | 60.8 (4.1) | 1.5 (3.8) | 40.4 | <0.0001 | 59.3 | <0.0001 |
| CFB (SE) | | | | (31.4, 49.4) | | (50.3, 68.3) | |

CFB, change from baseline; CI, confidence interval; FSH, follicle-stimulating hormone; LH, luteinizing hormone; LS, least squares; SD, standard deviation; SE, standard error; SHBG, sex hormone binding globulin.

TABLE D

| | | | | | | |
|---|---|---|---|---|---|---|
| LS | LS | | LS Mean | | LS Mean | |
| Mean % CFB | Mean % CFB | LS | Difference | | Difference | |
| or CFB (SE) | or CFB (SE) | Mean % CFB | Resmetirom | | Resmetirom | |
| Resmetirom | Resmetirom | or CFB (SE) | 80 mg from | | 100 mg | |
| 80 mg | 100 mg | Placebo | PBO | | from PBO | |
| (n = 322) | (n = 323) | (n = 321) | (95% CI) | p-value | (95% CI) | p-value |

FT3, ng/L

| | | | | | | | |
|---|---|---|---|---|---|---|---|
| no. | 279 | 265 | 286 | | | | |
| Baseline | 3.0 (0.41) | 3.0 (0.48) | 3.0 (0.41) | | | | |
| mean (SD) | | | | | | | |
| Week 52 | −0.01 (0.030) | −0.08 (0.031) | −0.03 (0.029) | 0.02 | 0.6058 | −0.05 | 0.1282 |
| CFB (SE) | | | | (−0.05, 0.08) | | (−0.12, 0.01) | |

FT3, ng/L (not on thyroxine)

| | | | | | | | |
|---|---|---|---|---|---|---|---|
| no. | 248 | 229 | 245 | | | | |
| Baseline | 3.0 (0.40) | 3.0 (0.42) | 3.1 (0.39) | | | | |
| mean (SD) | | | | | | | |
| Week 52 | −0.01 (0.032) | −0.08 (0.033) | −0.02 (0.031) | 0.01 | 0.7964 | −0.06 | 0.0926 |
| CFB (SE) | | | | (−0.06, 0.08) | | (−0.13, 0.01) | |

FT3, ng/L (thyroxine-treated)

| | | | | | | | |
|---|---|---|---|---|---|---|---|
| no. | 31 | 36 | 41 | | | | |
| Baseline | 2.7 (0.38) | 2.8 (0.72) | 2.8 (0.41) | | | | |
| mean (SD) | | | | | | | |
| Week 52 | 0.04 (0.089) | −0.03 (0.081) | −0.02 (0.079) | 0.05 | 0.5806 | −0.01 | 0.9161 |
| CFB (SE) | | | | (0.13, 0.23) | | (−0.19, 0.17) | |

FT4, ng/dL

| | | | | | | | |
|---|---|---|---|---|---|---|---|
| no. | 279 | 265 | 286 | | | | |
| Baseline | 1.1 (0.19) | 1.1 (0.21) | 1.1 (0.17) | | | | |
| mean (SD) | | | | | | | |
| Week | −13.9 (0.96) | −18.1 (0.97) | 2.6 (0.92) | −16.6 | <0.0001 | −20.7 | <0.0001 |
| 52 % CFB | | | | (−18.6, −14.5) | | (−22.8, −18.6) | |
| (SE) | | | | | | | |
| Week 52 | −0.17 (0.011) | −0.22 (0.011) | 0.02 (0.010) | −0.19 | <0.0001 | −0.24 | <0.0001 |
| CFB (SE) | | | | (−0.21, −0.16) | | (−0.26, −0.21) | |

FT4, ng/dL (not on thyroxine)

| | | | | | | | |
|---|---|---|---|---|---|---|---|
| no. | 248 | 229 | 245 | | | | |
| Baseline | 1.1 (0.18) | 1.1 (0.18) | 1.1 (0.16) | | | | |
| mean (SD) | | | | | | | |
| Week | −13.8 (0.97) | −17.6 (1.0) | 2.5 (0.95) | −16.3 | <0.0001 | −20.0 | <0.0001 |
| 52 % CFB | | | | (−18.4, −14.2) | | (−22.2, −17.9) | |
| (SE) | | | | | | | |
| Week 52 | −0.16 (0.011) | −0.21 (0.011) | 0.02 (0.010) | −0.18 | <0.0001 | −0.23 | <0.0001 |
| CFB (SE) | | | | (−0.20, −0.16) | | (−0.25, −0.20) | |

FT4, ng/dL (thyroxine-treated)

| | | | | | | | |
|---|---|---|---|---|---|---|---|
| no. | 31 | 36 | 41 | | | | |
| Baseline | 1.3 (0.23) | 1.2 (0.31) | 1.2 (0.21) | | | | |
| mean (SD) | | | | | | | |
| Week | −14.0 (3.6) | −20.6 (3.3) | 3.8 (3.1) | −17.9 | <0.0001 | −24.4 | <0.0001 |
| 52 % CFB | | | | (−25.2, −10.5) | | (−31.6, −17.3) | |
| (SE) | | | | | | | |
| Week 52 | −0.18 (0.041) | −0.26 (0.037) | 0.02 (0.036) | −0.21 | <0.0001 | −0.29 | <0.0001 |
| CFB (SE) | | | | (−0.29, −0.12) | | (−0.37, −0.21) | |

rT3, ng/dL

| | | | | | | | |
|---|---|---|---|---|---|---|---|
| no. | 278 | 269 | 282 | | | | |
| Baseline | 18.5 (5.4) | 19.2 (6.2) | 18.4 (5.6) | | | | |
| mean (SD) | | | | | | | |
| Week 52 | −4.6 (0.31) | −5.1 (0.32) | 0.17 (0.30) | −4.73 | <0.0001 | −5.2 | <0.0001 |
| CFB (SE) | | | | (−5.4, −4.1) | | (−5.9, −4.6) | |

rT3, ng/dL (not on thyroxine)

| | | | | | | | |
|---|---|---|---|---|---|---|---|
| no. | 247 | 232 | 244 | | | | |
| Baseline | 18.3 (5.3) | 18.7 (5.7) | 18.3 (5.6) | | | | |
| mean (SD) | | | | | | | |
| Week 52 | −4.5 (0.33) | −4.9 (0.34) | 0.19 (0.33) | −4.5 | <0.0001 | −5.1 | <0.0001 |
| CFB (SE) | | | | (−5.4, −3.9) | | (−5.9, −4.8) | |

rT3, ng/dL (thyroxine-treated)

| | | | | | | | |
|---|---|---|---|---|---|---|---|
| no. | 31 | 37 | 38 | | | | |
| Baseline | 20.7 (6.1) | 22.2 (8.1) | 19.1 (5.6) | | | | |
| mean (SD) | | | | | | | |
| Week 52 | −5.1 (0.94) | −6.3 (0.85) | −0.01 (0.84) | −5.1 | <0.0001 | −6.3 | <0.0001 |
| CFB (SE) | | | | (−7.1, −3.1) | | (−8.2, −4.4) | |

FT3/rT3

| | | | | | | | |
|---|---|---|---|---|---|---|---|
| no. | 278 | 269 | 282 | | | | |
| Baseline | 0.18 (0.058) | 0.17 (0.054) | 0.18 (0.058) | | | | |
| mean (SD) | | | | | | | |
| Week 52 | 0.06 (0.004) | 0.06 (0.004) | 0 (0.004) | 0.06 | <0.0001 | 0.07 | <0.0001 |
| CFB (SE) | | | | (0.05, 0.07) | | (0.06, 0.08) | |

FT3/rT3 (not on thyroxine)

| | | | | | | | |
|---|---|---|---|---|---|---|---|
| no. | 247 | 232 | 244 | | | | |
| Baseline | 0.18 (0.057) | 0.18 (0.051) | 0.18 (0.057) | | | | |
| mean (SD) | | | | | | | |
| Week 52 | 0.06 (0.005) | 0.06 (0.005) | 0 (0.005) | 0.06 | <0.0001 | 0.07 | <0.0001 |
| CFB (SE) | | | | (0.05, 0.07) | | (0.06, 0.08) | |

FT3/rT3 (thyroxine-treated)

| | | | | | | | |
|---|---|---|---|---|---|---|---|
| no. | 31 | 37 | 38 | | | | |
| Baseline | 0.14 (0.051) | 0.14 (0.058) | 0.16 (0.065) | | | | |
| mean (SD) | | | | | | | |
| Week 52 | 0.05 (0.011) | 0.06 (0.010) | −0.01 (0.010) | 0.06 | <0.0001 | 0.06 | <0.0001 |
| CFB (SE) | | | | (0.04, 0.09) | | (0.04, 0.09) | |

TSH, mIU/L

| | | | | | | | |
|---|---|---|---|---|---|---|---|
| no. | 279 | 265 | 286 | | | | |
| Baseline | 2.0 (1.1) | 2.1 (1.2) | 2.0 (1.1) | | | | |
| mean (SD) | | | | | | | |
| Week 52 | −0.28 (0.059) | −0.20 (0.060) | −0.10 (0.057) | −0.18 | 0.0057 | −0.10 | 0.1290 |
| CFB (SE) | | | | (−0.31, −0.05) | | (−0.23, 0.03) | |

TSH, mIU/L (not on thyroxine)

| | | | | | | | |
|---|---|---|---|---|---|---|---|
| no. | 248 | 229 | 245 | | | | |
| Baseline | 2.0 (1.0) | 2.0 (1.1) | 1.9 (0.98) | | | | |
| mean (SD) | | | | | | | |
| Week 52 | −0.23 (0.056) | −0.20 (0.058) | −0.08 (0.055) | −0.15 | 0.0143 | −0.12 | 0.0554 |
| CFB (SE) | | | | (−0.27, −0.03) | | (−0.24, 0) | |

TSH, mIU/L (thyroxine-treated)

| | | | | | | | |
|---|---|---|---|---|---|---|---|
| no. | 31 | 36 | 41 | | | | |
| Baseline | 2.0 (1.9) | 2.6 (1.5) | 2.2 (1.8) | | | | |
| mean (SD) | | | | | | | |
| Week 52 | −0.63 (0.27) | −0.13 (0.25) | −0.22 (0.24) | −0.41 | 0.1494 | 0.09 | 0.7507 |
| CFB (SE) | | | | (−0.97, 0.15) | | (−0.46, 0.63) | |

TBG, mg/L

| | | | | | | | |
|---|---|---|---|---|---|---|---|
| no. | 269 | 261 | 283 | | | | |
| Baseline | 24.4 (7.3) | 24.5 (8.1) | 24.3 (8.1) | | | | |
| mean (SD) | | | | | | | |
| Week 52 | −0.32 (0.45) | −0.60 (0.45) | 1.6 (0.43) | −1.9 | 0.0001 | −2.2 | <0.0001 |
| CFB (SE) | | | | (−2.8, −0.91) | | (−3.1, −1.2) | |

TBG, mg/L (not on thyroxine)

| | | | | | | | |
|---|---|---|---|---|---|---|---|
| no. | 238 | 225 | 244 | | | | |
| Baseline | 24.4 (7.5) | 24.1 (7.7) | 23.8 (7.7) | | | | |
| mean (SD) | | | | | | | |
| Week 52 | −0.19 (0.47) | −0.21 (0.49) | 1.6 (0.45) | −1.8 | 0.0007 | −1.8 | 0.0007 |
| CFB (SE) | | | | (−2.8, −0.74) | | (−2.8, −0.74) | |

TBG, mg/L (thyroxine-treated)

| | | | | | | | |
|---|---|---|---|---|---|---|---|
| no. | 31 | 36 | 39 | | | | |
| Baseline | 24.9 (5.8) | 26.6 (9.8) | 27.5 (9.7) | | | | |
| mean (SD) | | | | | | | |
| Week 52 | −1.1 (1.4) | −3.1 (1.3) | 1.5 (1.3) | −2.6 | 0.0924 | −4.6 | 0.0019 |
| CFB (SE) | | | | (−5.5, 0.43) | | (−7.5, −1.8) | |

CFB, change from baseline; CI, confidence interval; LS, least squares; rT3, reverse triiodothyronine; SE, standard error; FT3, free triiodothyronine; FT4, free thyroxine; TBG, thyroxine binding globulin; TSH, thyroid-stimulating hormone.

### EQUIVALENTS

While the present invention has been described in conjunction with the specific embodiments set forth above, many alternatives, modifications and other variations thereof will be apparent to those of ordinary skill in the art. All such alternatives, modifications, and variations are intended to fall within the spirit and scope of the present invention.

What is claimed is:

1. A method of treating or preventing a liver disorder in a subject, comprising:
(a) performing a baseline biomarker test on a biological sample obtained from the subject;
(b) administering a first dose of a thyroid hormone receptor-β (THR-β) agonist to the subject;
(c) performing a benchmark biomarker test on a biological sample obtained from the subject;
(d) performing a result assessment, thereby determining a second dose of the THR-β agonist; and
(e) administering the second dose of the THR-β agonist to the subject.

2. The method of claim 1, wherein the THR-β agonist is a selective THR-β agonist.

3. The method of claim 1, wherein the THR-β agonist is Compound A.

4. The method of claim 1, wherein the liver disorder is a fatty liver disease.

5. The method of claim 1, wherein the liver disorder is non-alcoholic fatty liver disease (NAFLD).

6. The method of claim 1, wherein the liver disorder is non-alcoholic steatohepatitis (NASH).

7. The method of claim 1, wherein the liver disorder is NASH with liver fibrosis.

8. The method of claim 1, wherein the subject is a human.

9. The method of claim 1, wherein the baseline biomarker test is performed before administering the first dose of the THR-β agonist.

10. The method of claim 1, wherein the baseline biomarker test comprises measuring an expression level of a biomarker.

11. The method of claim 1, wherein the baseline biomarker test comprises measuring an expression level of a sex hormone.

12. The method of claim 11, wherein the sex hormone is at least one selected from the group consisting of estradiol, follicle-stimulating hormone (FSH), luteinizing hormone (LH), testosterone, free testosterone, and sex hormone-binding globulin (SHBG).

13. The method of claim 12, wherein the baseline biomarker test comprises measuring the expression level of SHBG.

14. The method of claim 1, wherein the baseline biomarker test further comprises measuring an expression level of a thyroid hormone.

15. The method of claim 14, wherein the thyroid hormone is at least one selected from the group consisting of free triiodothyronine (FT3), reverse triiodothyronine (RT3), bound T3, total T3, free thyroxine (FT4), bound T4, total T4, or thyroid-stimulating hormone (TSH), and thyroxine binding globulin (TBG).

16. The method of claim 1, wherein the baseline biomarker test further comprises measuring an expression level of FT4.

17. The method of claim 1, wherein the baseline biomarker test comprises measuring: (i) an expression level of SHBG, and (ii) an expression level of FT4.

18. The method of claim 3, wherein the first dose of the Compound A is administered to the subject daily.

19. The method of claim 3, wherein the first dose of the Compound A is a daily dose of:
about 40±10 mg, about 40±5 mg, about 40±4 mg, about 40±3 mg, about 40±2 mg, or about 40±1 mg;
about 60±20 mg, about 60±10 mg, about 60±5 mg, about 60±4 mg, about 60±3 mg, about 60±2 mg, or about 60±1 mg;
about 80±20 mg, about 80±10 mg, about 80±5 mg, about 80±4 mg, about 80±3 mg, about 80±2 mg, or about 80±1 mg;
about 100±30 mg, about 100±20 mg, about 100±10 mg, about 100±5 mg, about 100±4 mg, about 100±3 mg, about 100±2 mg, or about 100±1 mg; or
about 120±40 mg, about 120±30 mg, about 120±20 mg, about 120±10 mg, about 120±5 mg, about 120±4 mg, about 120±3 mg, about 120±2 mg, or about 120±1 mg.

20. The method of claim 3, wherein a pharmaceutical composition comprising the first dose of the Compound A is administered to the subject.

21. The method of claim 3, wherein the first dose of the Compound A is administered to the subject for about a week or longer, about two weeks or longer, about three weeks or longer, about one month or longer, about two months or longer, about three months or longer, about four months or longer, about five months or longer, about six months or longer, about seven months or longer, about eight months or longer, about nine months or longer, about ten months or longer, about 11 months or longer, or about 12 months or longer.

22. The method of claim 3, wherein the first dose of the Compound A is administered to the subject for about one year or longer, for about two years or longer, for about three years or longer, for about four years or longer, for about five years or longer, for about six years or longer, for about seven years or longer, for about eight years or longer, for about nine years or longer, or for about ten years or longer.

23. The method of claim 3, wherein the first dose of the Compound A is administered to the subject until the second dose of the Compound A is determined.

24. The method of claim 3, wherein the benchmark biomarker test is performed after administering the first dose of the Compound A.

25. The method of claim 1, wherein the benchmark biomarker test comprises measuring an expression level of a sex hormone.

26. The method of claim 25, wherein the benchmark biomarker test comprises measuring the expression level of SHBG.

27. The method of claim 1, wherein the benchmark biomarker test further comprises measuring an expression level of a thyroid hormone.

28. The method of claim 1, wherein the benchmark biomarker test further comprises measuring an expression level of FT4.

29. The method of claim 1, wherein the benchmark biomarker test comprises measuring: (i) an expression level of SHBG, and (ii) the expression level of FT4.

30. The method of claim 1, wherein the result assessment comprises comparing a result of the benchmark biomarker test and a result of the baseline biomarker test.

31. The method of claim 1, wherein the result assessment comprises:
(i) determining that the second dose is lower than the first dose, if the benchmark biomarker test results in an expression level of SHBG at a high exposure threshold or higher, as compared to the baseline biomarker test;
(ii) determining that the second dose is higher than the first dose, if the benchmark biomarker test results in an expression level of SHBG lower than a target engagement threshold, as compared to the baseline biomarker test; and/or
(iii) determining that the second dose is the same as the first dose, if the benchmark biomarker test results in an expression level of SHBG lower than the high exposure threshold, and at the target engagement threshold or higher, as compared to the baseline biomarker test.

32. The method of claim 1, wherein:
the benchmark biomarker test results in an expression level of SHBG that is higher, as compared to the baseline biomarker test, by about 350% or greater, about 360% or greater, about 370% or greater, about 380% or greater, about 390% or greater, about 400% or greater, about 410% or greater, about 420% or greater, about 430% or greater, about 440% or greater, about 450% or greater, about 460% or greater, about 470% or greater, about 480% or greater, about 490% or greater, about 500% or greater, about 510% or greater, about 520% or greater, about 530% or greater, about 540% or greater, about 550% or greater, about 560% or greater, about 570% or greater, about 580% or greater, about 590% or greater, or about 600% or greater; and
the second dose is determined to be lower than the first dose by about 5 mg, about 10 mg, about 15 mg, about 20 mg, about 25 mg, about 30 mg, about 35 mg, or about 40 mg.

33. The method of claim 1, wherein:
the benchmark biomarker test results in an expression level of SHBG that is higher, as compared to the baseline biomarker test, by about 40% or less, about 50% or less, about 60% or less, about 70% or less, about 80% or less, about 90% or less, about 100% or less, about 110% or less, about 120% or less, about 130% or less, about 140% or less, about 150% or less, about 160% or less, about 170% or less, about 180% or less, about 190% or less, about 200% or less, about 210% or less, about 220% or less, about 230% or less, about 240% or less, about 250% or less, about 260% or less, about 270% or less, about 280% or less, about 290% or less, or about 300% or less; and
the second dose is determined to be higher than the first dose by about 5 mg, about 10 mg, about 15 mg, about 20 mg, about 25 mg, about 30 mg, about 35 mg, or about 40 mg.

34. The method of claim 1, wherein the result assessment comprises:
(i) determining that the second dose is lower than the first dose, if the benchmark biomarker test results in an expression level of FT4 at a high exposure threshold or lower, as compared to the baseline biomarker test;
(ii) determining that the second dose is higher than the first dose, if the benchmark biomarker test results in an expression level of FT4 higher than a target engagement threshold, as compared to the baseline biomarker test; and/or
(iii) determining that the second dose is the same as the first dose, if the benchmark biomarker test results in an expression level of FT4 higher than the high exposure threshold, and at the target engagement threshold or lower, as compared to the baseline biomarker test.

35. The method of claim 1, wherein:
the benchmark biomarker test results in an expression level of FT4 that is lower, as compared to the baseline biomarker test, by about 19% or greater, about 20% or greater, about 21% or greater, about 22% or greater, about 23% or greater, about 24% or greater, about 25% or greater, about 26% or greater, about 27% or greater, about 28% or greater, about 29% or greater, about 30% or greater, about 35% or greater, or about 40% or greater; and
the second dose is determined to be lower than the first dose by about 5 mg, about 10 mg, about 15 mg, about 20 mg, about 25 mg, about 30 mg, about 35 mg, or about 40 mg.

36. The method of claim 1, wherein:
the benchmark biomarker test results in an expression level of FT4 that is lower, as compared to the baseline biomarker test, by about 5% or less, about 6% or less, about 7% or less, about 8% or less, about 9% or less, about 10% or less, about 11% or less, about 12% or less, about 13% or less, about 14% or less, about 15% or less, or about 16% or less; and
the second dose is determined to be higher than the first dose by about 5 mg, about 10 mg, about 15 mg, about 20 mg, about 25 mg, about 30 mg, about 35 mg, or about 40 mg.

37. The method of claim 3, wherein the second dose of the Compound A is administered to the subject daily.

38. The method of claim 3, wherein the second dose of the Compound A is a daily dose of:
about 40±10 mg, about 40±5 mg, about 40±4 mg, about 40±3 mg, about 40±2 mg, or about 40±1 mg;
about 60±20 mg, about 60±10 mg, about 60±5 mg, about 60±4 mg, about 60±3 mg, about 60±2 mg, or about 60±1 mg;
about 80±20 mg, about 80±10 mg, about 80±5 mg, about 80±4 mg, about 80±3 mg, about 80±2 mg, or about 80±1 mg;
about 100±30 mg, about 100±20 mg, about 100±10 mg, about 100±5 mg, about 100±4 mg, about 100±3 mg, about 100±2 mg, or about 100±1 mg; or
about 120±40 mg, about 120±30 mg, about 120±20 mg, about 120±10 mg, about 120±5 mg, about 120±4 mg, about 120±3 mg, about 120±2 mg, or about 120±1 mg.

39. The method of claim 1, wherein the method further comprises performing one or more baseline tests and/or benchmark tests on one or more metabolic factors.

40. A method of treating or preventing a liver disorder in a subject, comprising:
(a) administering a low-age dose of a THR-B agonist to the subject, wherein the subject has an age younger than a dose-adjustment age threshold; and/or
(b) administering a high-age dose of the THR-B agonist to the subject, wherein the subject has an age at a dose-adjustment age threshold or older.

41. The method of claim 40, wherein the THR-β agonist is a selective THR-β agonist.

42. The method of claim 40, wherein the THR-B agonist is Compound A.

43. The method of claim 40, wherein the liver disorder is a fatty liver disease.

44. The method of claim 40, wherein the liver disorder is non-alcoholic fatty liver disease (NAFLD).

45. The method of claim 40 wherein the liver disorder is non-alcoholic steatohepatitis (NASH).

46. The method of claim 40, wherein the liver disorder is NASH with liver fibrosis.

47. The method of claim 40, wherein the subject is a human.

48. The method of claim 40, wherein the subject has the age younger than the dose-adjustment age threshold, and the THR-B agonist is administered to the subject at the low-age dose.

49. The method of claim 40, wherein the subject has the age at the dose-adjustment age threshold or older, and the THR-B agonist is administered to the subject at the high-age dose.

50. The method of claim 40, wherein the dose-adjustment age threshold is about 50 years old, about 55 years old, about 60 years old, about 65 years old, about 70 years old, about 75 years old, or about 80 years old.

51. The method of claim 40, wherein the dose-adjustment age threshold is about 60 years old, about 61 years old, about 62 years old, about 63 years old, about 64 years old, about 65 years old, about 66 years old, about 67 years old, about 68 years old, about 69 years old, or about 70 years old.

52. The method of claim 40, wherein the dose-adjustment age threshold is about 65 years old.

53. The method of claim 42, wherein the low-age dose is a daily dose of about 100±30 mg, about 100±20 mg, about 100±10 mg, about 100±5 mg, about 100±4 mg, about 100±3 mg, about 100±2 mg, or about 100±1 mg.

54. The method of claim 42, wherein the low-age dose is about 100 mg.

55. The method of claim 42, wherein the high-age dose is lower than the low-age dose.

56. The method of claim 42, wherein the high-age dose is lower than the low-age dose by about 5 mg, about 10 mg, about 15 mg, about 20 mg, about 25 mg, about 30 mg, about 35 mg, or about 40 mg.

57. The method of claim 42, wherein the high-age dose is lower than the low-age dose by about 20 mg.

58. The method of claim 42, wherein the high-age dose is a daily dose of:
about 60±20 mg, about 60±10 mg, about 60±5 mg, about 60±4 mg, about 60±3 mg, about 60±2 mg, or about 60±1 mg;
about 80±20 mg, about 80±10 mg, about 80±5 mg, about 80±4 mg, about 80±3 mg, about 80±2 mg, or about 80±1 mg; or
about 100±30 mg, about 100±20 mg, about 100±10 mg, about 100±5 mg, about 100±4 mg, about 100±3 mg, about 100±2 mg, or about 100±1 mg.

59. The method of claim 42, wherein the high-age dose is a daily dose of about 60 mg, about 80 mg, or about 100 mg.

60. The method of claim 42, wherein the subject has the age younger than 65 years old, and the Compound A is administered to the subject at a daily dose of about 100±30 mg, about 100±20 mg, about 100±10 mg, about 100±5 mg, about 100±4 mg, about 100±3 mg, about 100±2 mg, or about 100±1 mg.

61. The method of claim 42, wherein the subject has the age at 65 years old or older, and the Compound A is administered to the subject at a daily dose of about 80±20 mg, about 80±10 mg, about 80±5 mg, about 80±4 mg, about 80±3 mg, about 80±2 mg, or about 80±1 mg.
